(19) United States
(12) Patent Application Publication
(10) Pub. No.: US 2009/0325867 A1
(43) Pub. Date: Dec. 31, 2009

(54) RECEPTOR-SPECIFIC TUMOUR NECROSIS FACTOR-RELATED APOPTOSIS-INDUCING LIGAND (TRAIL) VARIANTS

(75) Inventors: Gerald Cohen, Leicester (GB); Marion MacFarlane, Leicester (GB); Michael Sutcliffe, Manchester (GB)

Correspondence Address: DAVID S. RESNICK, NIXON PEABODY LLP, 100 SUMMER STREET, BOSTON, MA, 02110-2131, US

(73) Assignees: MEDICAL RESEARCH COUNCIL, London (GB); UNIVERSITY OF LEICESTER, Leicester (GB)

(21) Appl. No.: 12/094,315

(22) PCT Filed: Nov. 29, 2006
(86) PCT No.: PCT/GB06/04461

§ 371 Date: Oct. 6, 2008

(30) Foreign Application Priority Data
Nov. 29, 2005 (GB) 0524316.7

(51) Int. Cl. A61K 38/17; C07K 14/705; C12N 5/02

(52) U.S. Cl. 514/12; 530/350; 435/375

(57) ABSTRACT

The invention relates to a tumour necrosis factor- (TNF-) related apoptosis-inducing ligand (TRAIL) which is capable of selectively signalling through death receptor 4 (DR4), comprising Y at position 189. Preferably the TRAIL further comprises 19 IL and/or 199V; preferably also 201R, 213W and 215D, and/or preferably further comprises 193S. The invention also relates to uses of such TRAIL mutants which are capable of selectively signalling through DR4 in the treatment of cancer, and in the manufacture of medicaments for use in treatment of cancer. Preferably the cancer is chronic lymphocytic leukaemia, mantle cell lymphoma or non-Hodgkin's lymphoma. The invention also relates to kits comprising same.

### FIELD OF THE INVENTION

The invention is in the field of tumour necrosis factor- (TNF-) related apoptosis-inducing ligand (TRAIL). In particular the invention relates to TRAIL receptor 1 (TRAIL-R1, sometimes called death receptor 4 (DR4)) selective TRAIL mutants.

### BACKGROUND OF THE INVENTION

TNF-related apoptosis-inducing ligand (TRAIL) is a transmembrane protein which can be cleaved at the cell surface to form a soluble ligand. TRAIL induces apoptosis by binding at TRAIL-R1 (Death Receptor-4 (DR4)) or TRAIL-R2 (Death Receptor-5 (DR5/TRICK2)). TRAIL induces apoptosis in cancer cell lines but not in normal cells. TRAIL is sometimes referred to as apoptosis-inducing ligand 2 (Apo2L).

Signalling via DR5 is important in cancer and cancer models, for example lung cancer, breast cancer, colon cancer and others. Signalling via DR4 is important in cancer and cancer models such as chronic lymphoid leukaemia (CLL).

Kelley et al (2005 J Biol Chem Volume 280, pages 2205-2212) disclose a study of receptor selective TRAIL mutants. This study is focused on the differences between the DR4 and DR5 activities. Various TRAIL mutants are studied, and it is concluded that DR5 may contribute more than DR4 to TRAIL induced apoptosis in cancer cells that express both death receptors. Ligand variants were selected using a phage display approach to have relative binding selectivity for DR4 or DR5. The action of these mutants was studied in various cancer cell lines and in normal hepatocytes. The results indicate that DR5 plays a more prominent role than DR4 in mediating apoptosis. Substitution of Tyr-189 with Ala in TRAIL is said to be important for DR4 selectivity since all four of the clones studied as DR4 selective variants each had this mutation. Furthermore, H264R and/or D267Q substitutions are said to give further selectivity against DR4 binding while providing modest improvements in DR5 affinity and bioactivity.

MacFarlane et al (2005 Cell Death and Differentiation vol 12 pages 773-782) discloses a wide ranging survey of the response of various cell lines and primary cells to signalling via TRAIL-R1 or TRAIL-R2. A principal teaching of this document is that TRAIL induced apoptosis in chronic lymphocytic leukaemic cells (CLL cells) proceeds predominantly via TRAIL-R1 in the presence of a histone deacetylase inhibitor (HDACi). Thus, to maximise therapeutic benefit, it is regarded as essential to ascertain whether a primary tumour signals via TRAIL-R1 or TRAIL-R2 prior to initiating therapy. Specific therapeutic combinations for treatment of CLL are proposed. Furthermore, it is disclosed that subtle alterations in different forms of TRAIL can alter their propensity to activate different TRAIL receptors. This clearly markedly alters their biological activities. It is taught that use of TRAIL targeting therapy will require prior in vitro testing to assess the sensitivities of different tumours to TRAIL-R1 or TRAIL-R2.

Kaufmann and Steensma (2005 Leukemia vol 19 pp 2195-2202) present a review of the biological function of TRAIL, the mechanism of cytotoxicity and the effects of TRAIL on various cells in vitro. Kaufmann and Steensma acknowledge that DR5 is considered to be the predominant receptor involved in TRAIL induced killing of tumour cells. They review evidence that has failed to validate the suggested toxicity of TRAIL to human hepatocytes, particularly by DR5 directed reagents. This reinforces the view in the art that DR5 signalling is still the best candidate for effective therapy. The review goes on to discuss the MacFarlane et al publication (ibid) at length (see above). Kaufmann and Steensma do not disclose any TRAILs.

WO 97/25428 discloses an Apo-2 ligand. This document describes cDNA and cloning of Apo-2 L. Fragments of Apo-2 L are described, and molecules having various levels of sequence identity with the Apo-2 L sequence are mentioned. In its broadest aspect, this document teaches an Apo-2 ligand having at least 80% homology to amino acids 114-281 of Apo-2 L, which can induce apoptosis in at least one type of mammalian cell. There are no teachings regarding the mutation of the Apo-2 L sequence. There is no teaching regarding the retention of function. There is no teaching regarding which residues may be varied, and which should be retained in order to retain Apo-2 L function. There is no teaching of direction of activity towards a particular receptor or sub type of receptor. Although this document could be seen to relate to myriad shortened sequence variants of Apo-2 L, there is no indication in the disclosure which of the variants might be active, or how to select them. Furthermore, there is no teaching of DR4 selectivity.

WO 99/36535 describes Apo-2 ligand polypeptides. Furthermore, fragments of those polypeptides are claimed such as polypeptides comprising amino acids 91-281 of Apo-2 L, or comprising amino acids 92-281 of Apo-2 L. Three specific mutations of the Apo-2 ligand are described. The mutations disclosed are D203A, D218A and D269A. Apart from these three specific mutations, there is no guidance given regarding the residues which should be retained for biological activity, and those which can be mutated. There is no discussion of said activity for a particular receptor or receptor sub type. There is no teaching of direction of activity to the DR4 receptor.

WO 2004/101608 describes a range of Apo-2 L receptor binding peptides. In particular, table 8 of this document discloses 91 different peptides which are said to be capable of binding Apo-2 L receptors. These range from approximately ten amino acids to approximately 25 amino acids in length. Furthermore, there is disclosure of particular cysteine related motifs which are preferably comprised by those peptides. Although the DR4 and DR5 receptors are mentioned, the focus is firmly on inhibition of interaction between Apo-2 L and those receptors. The overall teaching of this document goes towards the inhibition of signalling by Apo-2 L by use of these inhibitory peptides. There is no teaching of new TRAIL mutants in this document. There is no teaching of how to produce signalling via DR4 in this document. There is no guidance regarding residues of TRAIL which could be important for signalling.

Kelley et al 2005 teach that Tyr 189 of TRAIL must be Ala for DR4 signalling.

The prior art suffers from the problem that most studies have been performed in cultured cell lines rather than primary cells.

The prior art emphasises the importance of DR5 signalling.

Prior art studies are focused on receptor binding, and present little or no data regarding biological functionality.

Fully wild type TRAIL gives good apoptotic cell death. However, levels are comparable in DR4 and DR5 backgrounds. This leads to problems of liver toxicity. Liver toxicity can be attributed to activation of TRAIL/apoptosis in hepatocytes. This signalling is believed to proceed via DR5. Problems have been encountered using wild-type TRAIL therapies.

The present invention seeks to overcome problem(s) associated with the prior art.

### SUMMARY OF THE INVENTION

The present invention is based on the surprising finding of specific TRAIL mutants which possess DR4 selectivity.

Prior art ligands which are said to possess DR4 specificity have been shown by the present inventors to be unexpectedly biologically inactive. This may be because the prior art data was focused on receptor binding studies rather than a biological assay of functionality. Nevertheless, it is an advantage of the present invention that TRAIL mutants possessing DR4 biological signalling capabilities are provided.

Furthermore, since liver toxicity in hepatocytes is considered to act predominantly via DR5, it is an advantage of the present invention that DR4 selective TRAIL mutants help to reduce or eliminate liver toxicity.

Prior art studies have been focused on the use of wild type TRAIL binding as a reference point. In the prior art, ‘selectivity’ for DR4 or DR5 has been used to describe an increased binding to DR4 or DR5 rather than a selective biological effect. As is shown by the present inventors, this binding does not always translate into actual biological activity. It is an advantage of the present invention that the biologically important residues in the TRAIL sequence have been defined with respect to DR4 signalling.

In the prior art, the Y189A mutation was said to be important to DR4 signalling. However, the present inventors found that TRAIL mutants with this mutation had no biological activity. Contrary to the prior art teachings, the present inventors teach that Y189 is important to DR4 signalling. Thus in one aspect the invention provides a TRAIL which is capable of selectively signalling through DR4, comprising Y at position 189.

In another aspect, the invention provides a TRAIL as described above further comprising 193S.

In another aspect, the invention provides a TRAIL as described above further comprising 191L.

In another aspect, the invention provides a TRAIL as described above further comprising 199V.

In another aspect, the invention provides a TRAIL as described above further comprising 201R, 213W and 215D, preferably in combination with 199V.

In another aspect, the invention provides a TRAIL as described above which comprises sequence corresponding to at least amino acids 95-281 of TRAIL.

In another aspect, the invention provides a TRAIL as described above further comprising a non-peptide polymer. Linkage of the TRAIL to a non-peptide polymer advantageously stabilises the TRAIL. The polymer may be any suitable polymer known in the art for this purpose. Preferably said polymer is selected from the group consisting of polyethylene glycol, polypropylene glycol and polyoxyalkylene.

The prior art focuses on DR5 as important in cancer signalling, and aims at the production of DR5 signal at the expense of DR4. However, the present inventors have appreciated the value of the DR4 signal contrary to the teachings of the prior art. Thus, in another aspect, the invention provides use of a TRAIL which is capable of selectively signalling through DR4 in the treatment of cancer. Preferably the cancer is a B-cell malignancy. Preferably the cancer is chronic lymphocytic leukaemia or mantle cell lymphoma or B-cell non-Hodgkin's lymphoma.

The present inventors have discovered that some cancers signal/respond via DR5 and some via DR4. Indeed, one reason why the prior art may place so much emphasis on DR5 is that it is mainly based on work with immortalised cell lines in vitro. By contrast, the present inventors present insights into the signalling events of primary cancer cells, and surprisingly find that these respond via DR4. Therefore, it is important to determine whether the disorder to be treated responds to DR4 or a different signal, and direct the therapy accordingly. Thus, in another aspect, the invention provides a method of treating a cancer in a subject by administration of TRAIL which is capable of selectively signalling through DR4, said method comprising determining if the target cells of said subject signal via DR4, wherein if said target cells do signal via DR4 then a TRAIL which is capable of selectively signalling through DR4 is administered. In another aspect, the invention relates to a method of aiding the diagnosis of a DR4-responsive disorder in a subject said method comprising determining if the target cells of said subject signal via DR4, wherein if said target cells do signal via DR4 then the diagnosis of a DR4-responsive disorder is confirmed.

The ‘target cells’ are those cells which is it clinically desirable to remove, kill, destroy or otherwise arrest or eliminate from the subject. Preferably such arrest or elimination is by induction of apoptosis. Preferably the target cells are the actual cells associated with or responsible for the disorder, such as the cancer/tumour cells.

In another aspect, the invention provides a method of treatment as described above, further comprising administering a sensitising agent. Preferably said sensitising agent is a HDAC inhibitor. Preferably said HDAC inhibitor is valproate, preferably sodium valproate.

In another aspect, the invention provides use of a TRAIL which is capable of selectively signalling through DR4 in the manufacture of a medicament for the prevention or treatment of cancer wherein the cells of said cancer signal through DR4. Preferably the invention provides use of a TRAIL which is capable of selectively signalling through DR4 in the manufacture of a medicament for cancer wherein the cells of said cancer signal through DR4. Preferably said cells signal primarily through DR4. Furthermore the invention provides a TRAIL which is capable of selectively signalling through DR4 for use in the treatment of cancer, preferably the cancer is CLL, MCL or NHL.

In another aspect, the invention provides a method for treatment of a B-cell malignancy in a subject, or a B-cell non-Hodgkin's lymphoma or mantle cell lymphoma or chronic lymphocytic leukaemia in a subject, the method comprising administering to said subject a TRAIL which is capable of selectively signalling tlirough DR4.

In another aspect, the invention provides a method as described above further comprising administering to said subject a histone deacetylase inhibitor (HDACi).

Preferably the TRAIL is administered after the HDACi is administered.

Preferably the TRAIL is administered at least 8 hours after the HDACi is administered.

Preferably the TRAIL is administered at least 16 hours after the HDACi is administered.

Preferably the HDACi is administered over a period of about 8-16 hours.

Preferably the TRAIL is administered over a period of about 4-8 hours, preferably about 4 hours.

Preferably multiple administrations of TRAIL and/or HDACi are made to said subject.

In another aspect, the invention provides a method of inducing formation of death inducing signalling complex (DISC) in a haematological malignant cell comprising contacting said cell with a histone deacetylase inhibitor (HDACi) and a TRAIL which is capable of selectively signalling through DR4.

In another aspect, the invention provides a method of inducing caspase activation in a haematological malignant cell comprising contacting said cell with a histone deacetylase inhibitor (HDACi) and a TRAIL which is capable of selectively signalling through DR4.

Preferably the HDACi is selected from the group consisting of depsipeptide, Trichostatin A (TSA) and valproate. Preferably the HDACi is valproate.

In another aspect, the invention provides a kit comprising a TRAIL which is capable of selectively signalling through DR4 and a sensitising agent.

In the methods, kits and uses as described above, preferably the TRAIL is a TRAIL as described herein.

Preferably the invention relates to TRAIL R1 (DR4) applications such as TRAIL R1 (DR4) selective TRAIL mutants.

### DETAILED DESCRIPTION OF THE INVENTION

TNF receptor apoptosis-inducing ligand (TRAIL) and its agonistic antibodies, which are currently in early clinical trials for treating various malignancies, induce apoptosis through triggering of either TRAIL-R1 or -R2. We now unequivocally demonstrate that primary cells from patients with certain lymphoid malignancies signal to apoptosis almost exclusively through TRAIL-R1. Thus we propose that no therapeutic benefit can be anticipated from treating such patients with agents currently in clinical trials that signal predominantly through TRAIL-R2, such as HGS-ETR2 (Human Genome Sciences) or Apo2L/TRAIL (Genentech). According to the present invention it is advantageous to determine whether primary cells from a particular tumor will signal via TRAIL-R1 or -R2. Such information provides a new rational approach to optimize TRAIL therapy.

Whereas many cancer cell lines are sensitive to TRAIL, most primary cells from patients, such as those with chronic lymphocytic leukemia (CLL) and B-cell non-Hodgkin's lymphoma, are resistant. Combination treatments with chemotherapeutic agents may be used to sensitize resistant cells to TRAIL-induced apoptosis. We show that histone deacetylase inhibitors (HDACi), such as depsipeptide, sensitize CLL cells to TRAIL-induced apoptosis by facilitating increased formation of the TRAIL DISC.

TRAIL induces apoptosis in a wide range of tumor cell lines but is non-toxic to most normal cells and tissues. Clinical trials have been initiated with HGS-ETR1 and HGS-ETR2 (Human Genome Sciences), selective agonistic antibodies for TRAIL-R1 and TRAIL-R2, respectively and also with Apo2L/TRAIL (Genentech). Although TRAIL can signal apoptosis via TRAIL-R1 or -R2, the majority of studies suggest that TRAIL-R2 is the primary receptor leading to cell death. However most of these studies have used cell lines; only a few used primary tumor cells from patients. Using antibodies and various TRAIL ligands in combination with cell lines, which signal predominantly through TRAIL-R1 (Ramos) or TRAIL-R2 (Jurkat), we propose that clinically relevant cells such as primary CLL cells signal predominantly through TRAIL-R1.

Using site-directed mutagenesis, we have now synthesized a series of TRAIL mutants that bind selectively to either TRAIL-R1 or -R2. The TRAIL-R1, but not TRAIL-R2, selective mutants induce apoptosis in primary CLL and mantle cell lymphoma (MCL) cells thereby demonstrating conclusively that these primary lymphoid cells signal through TRAIL-R1 and not through TRAIL-R2.

We disclose that CLL cells signal via TRAIL-R1 to induce apoptosis. This is of major importance to the form of TRAIL that should be used clinically. Apo2L/TRAIL of the prior art signals primarily through TRAIL-R2 and will be useless for CLL and other malignancies that signal through R1.

We synthesized supposed TRAIL-R1 (DR4) selective mutants from the prior art and found them to be inactive. The prior art conclusions regarding the importance of TRAIL-R1 are wrong. We have now modified the structure and produced mutants that act selectively on TRAIL-R1. These DR4 selective mutants are active on CLL cells and a TRAIL-R2 selective mutant is inactive. This conclusively proves the importance of TRAIL-R1 (DR4) signalling in CLL as an exemplary DR4 signalling malignancy.

TRAIL-R1 is more important than TRAIL-R2 in primary tumors. Thus, the invention finds application in the treatment of tumours or malignancies which signal via DR4 (TRAIL-R1).

TRAIL

The sequence of wild type TRAIL is the reference sequence used herein in the description of the sequences of the TRAIL mutants of the present invention. The wild type TRAIL sequence is preferably human TRAIL, preferably accession number NM_{—}003810. References to TRAIL preferably include soluble TRAIL (sometimes referred to as ‘sTRAIL’). Soluble TRAILs or TRAIL mutants are preferred since they are advantageously easier to prepare and/or administer. Soluble TRAIL (sTRAIL) refers to aa95-281 of TRAIL; aa1-94 represent the transmembrane domain and therefore aa95-281 is the soluble TRAIL. Typically references to ‘TRAIL’ or ‘TRAIL mutants’ of the invention refer to sTRAIL or aa95-281 with the relevant substitutions as will be apparent from the context.

Length

The TRAIL mutants of the present invention may vary in length for example at their N-termini. His-TRAIL encodes amino acids 95-281 together with an N-terminal His tag. Apo2L comprises amino acids 114-281. There is no experimental evidence to suggest that the short region at the N-terminus of TRAIL (eg. amino acids 95-114) plays any key role in binding of TRAIL to its receptors. Thus, TRAIL mutants of the present invention may include amino acids in this N-terminal region, or may not, depending upon operator choice. The key teaching of the invention lies in the amino acid changes made to the regions of TRAIL important for DR4 specificity. Consistent with this, we have mutated the same residues proposed by Kelley et al (2005 J Biol Chem Volume 280, pages 2205-2212) to produce a TRAIL-R2 specific mutant of Apo2L and this has resulted in a similarly TRAIL-R2-specific form of His-TRAIL (TRAIL.R2-6), demonstrating that the teachings of the present invention regarding mutations can be applied in different TRAIL contexts according to the present invention.

Preferably TRAIL mutants according to the present invention comprise amino acid sequence corresponding to at least 25aa of TRAIL, preferably at least 50aa, preferably at least 100aa, preferably at least 150aa, preferably at least 161aa, preferably at least 167aa, preferably at least 190aa, preferably at least 200aa, preferably at least 240aa, preferably the full 281aa. When the mutants of the invention comprise amino acid sequence amounting to less than the full length 281aa of TRAIL, preferably the sequence is firm the C-terminus of the full 281aa of TRAIL, preferably from the soluble trimeric TRAIL sequence. TRAIL mutants comprising less than the full length are discussed in more detail below.

The crystal structure of soluble trimeric TRAIL is based on residues 114-281, while the crystal structure of TRAIL in complex with DR5 has shown that residues 120-135 and 146-281 in each TRAIL monomer are critical for the interaction with DR5. Thus, preferably a TRAIL according to the invention comprises at least residues 120-135 and 146-281. Preferably the minimal length for a TRAIL-R1/2 mutant according to the present invention is residues 120-281. Preferably the TRAIL mutants of the invention comprise aa120 to aa281 of TRAIL; preferably aa114 to aa281, preferably aa95 to aa281, preferably aa92 to aa281, preferably aa91 to aa281, or full length TRAIL. Clearly this discussion of length with reference to TRAIL 1-281 sequence applies only to the TRAIL part of a polypeptide according to the present invention ie. any tags or fusions are to be in addition to the TRAIL residues mentioned. Furthermore, when discussing ‘length’, to say that the ligand comprises for example 120-281 of TRAIL does not imply that the ligand comprises exactly that amino acid sequence—attention must still be paid to the key feature of the present invention ie. the particular amino acid substitutions made. The discussions of length must be interpreted in this context. As a default, unless the context gives a different meaning, preferably TRAIL amino acid residues which are not specified are as for wild-type human TRAIL. The same applies to any nucleic acids encoding TRAIL polypeptides of the invention.

Numbering of the residues of the TRAIL mutants discussed herein follows the established conventions in the art, ie. the numbering used corresponds to positions on the wild type TRAIL sequence.

The term ‘fragments thereof’ will be understood to relate only to signalling-competent fragments of the specified TRAIL mutant. The question whether a fragment (or ligand) is signalling competent is easily determined with reference to the tests set out herein for DR4 signalling/death induction. A signalling competent fragment need not possess the full activity of a fuller length TRAIL but must possess the same qualitative activity ie. must be DR4 selective/specific. The same criteria for DR4 selectivity/specificity apply to fragments of the TRAIL mutant as apply to the ligands themselves. In any case a fragment must comprise amino acid sequence corresponding to at least 25 aa of TRAIL.

TRAIL Mutants: Sequence Substitutions

The term ‘mutant’ as used herein has its natural meaning in the art. Specifically, a ‘TRAIL mutant’ is a TRAIL which differs from the wild-type TRAIL at at least one position of its sequence, preferably amino acid sequence. Individual mutants and their mutation(s) are discussed in detail herein.

The two most preferred TRAIL mutants disclosed herein possess five and four amino acid changes relative to the wild type TRAIL ligand.

When only a certain two of these amino acid changes are made (TRAIL.R1-2: Y213W;S215D), the same activity is not achieved. Thus, this TRAIL mutant is specifically disclaimed from the present invention.

Specificity may be achieved with an alternative two amino acid mutant TRAIL (N199V;K201R), with a three amino acid mutant TRAIL (Q193S;N199V;K201R).

When only the single N199V substitution at amino acid 199 is made (TRAIL N199V), specificity is advantageously conferred. Thus, this single mutant TRAIL is a preferred TRAIL of the present invention.

Y189

In addition to the prior art teaching that Y189A is important to DR4 specificity, TRAIL-R1 and -R2 (DR4 and 5) appear to be conserved in the region of the receptor which interacts with Y189 of TRAIL. Consequently, without knowledge of the present invention it is difficult to predict mutations in this region of TRAIL that could be selective.

Small hydrophobic: From our structural model, it appears that Y189A will result in weaker protein-protein interactions to roughly the same extent with -R1 and -R2. This is consistent with the experimental data presented herein.

Larger hydrophobic: Phe or Leu here would be expected to weaken the protein-protein interaction (loss of hydrogen bond, but hydrophobic interaction maintained), but to affect -R1 and -R2 binding to roughly the same extent.

As noted above, the prior art teaches that the Y189A substitution is central to DR4 selectivity. By contrast, the present inventors discovered that Y189A mutants had no functional activity in biologically relevant DR4 signalling. In fact, substitution at amino acid 189 to a small, polar residue (Y189Q) or non-polar residue (Y189A) results in a significant loss of activity for DR4. Thus preferably TRAIL mutants of the present invention do not comprise Y189Q or Y189A. Preferably TRAIL mutants of the present invention comprise Y189 ie. are wild-type at position 189.

In the prior art, the choice of particular mutations is dominated by the teachings of Kelley et al (corresponding to TRAIL.R1-6 herein). However, we have shown that these six amino acid substitutions result in an inactive form of the ligand and that activity and specificity are only retained in alternative mutants such as the preferred four or five amino acid TRAIL mutant species of the invention (eg. TRAIL.R1-4/5).

R191

From our model (TRAIL/TRAIL-R1) we predict that amino acid 191 is critical for the interaction of TRAIL with TRAIL-R1. To assess this, we have mutated R191L (making it hydrophobic), and then depending on the effect of this single substitution we then make systematic mutations of this amino acid. For example, R191Q (polar and shorter, but not charged), R191A (non polar and small), and R191E (charge reversal). From this, a comparison of R191Q and R191E enables determination of the effects of charge.

R191 binds to ASP (67) in TRAIL R2 forming as salt bridge (2.6A) which is important for stabilisation of TRAIL R2. In this application, the residue numbering used for the TRAIL-R2 receptor is identical to that used in the reference by Hymovitz et al (Molecular Cell Volume 4, 563-571, 1999) and refers to TRAIL-R2 without its N-terminal signal peptide. To further clarify this point, in mature TRAIL-R2 Asp 67 corresponds to Asp 120 in the full-length TRAIL-R2 sequence, including the N-terminal signal peptide. R191 of TRAIL also forms an indirect hydrogen bond through an intermediate water molecule with Ser(68) in TRAIL R2. Since this serine residue is conserved in TRAIL R1, this indirect hydrogen bond could be effected in R1 as well if desired. Therefore, mutations at this site will enhance DR4. Preferred are mutations introducing negative charge at this site.

Various mutations at R191 may be made according to the present invention. Depending on the substitution made, different effects may be achieved. Some of these are discussed below. Preferably R191 is mutated to a residue other than Ser.

R191L introduces hydrophobicity.

R191Q renders it polar and shorter, but not charged.

R191A removes the side chain completely.

R191E results in a charge reversal. This is a chemically strong effect. Comparison of R191Q and R191E enables determination of the effects of charge in more detail.

Thus in summary we teach that substitution of R191 to either a hydrophobic (Leu) or charge reversed (Glu) residue should confer degrees of specificity for TRAIL-R1.

By way of explanation, charge reversal: R191E/D may have adverse interaction with D67 of TRAIL-R2 and may also enhance interaction with TRAIL-R1 via favourable interaction with K67 of TRAIL-R1. In this application, the residue numbering used for the TRAIL-R1 receptor is one that refers to the TRAIL-R1 sequence without its N-terminal signal peptide as shown in FIG. 3A and in the reference by Hymovitz et al. To further clarify this point, in mature TRAIL-R1 K67 corresponds to K171 in the full-length TRAIL-R1 sequence. R191E is preferred.

Polar, non-charged: R191Q/N may be less effective than R191E, but could remove/weaken interaction with D67 of TRAIL-R2 and may thereby enhance binding to TRAIL-R1.

Hydrophobic: R191L/V/A may weaken protein-protein interactions, more so in TRAIL-R2 than TRAIL-R1, and may therby confer specificity for TRAIL-R1. Preferred is R191L.

A preferred R191 mutation of the invention is R191E.

The specific single mutant TRAIL R191L confers DR4 specificity and so this single mutant TRAIL is a preferred TRAIL mutant of the invention.

N199

Substitution of amino acid N199 from a polar residue (Asn) to a small, non-polar residue (eg. Val/Ala/Leu) confers specificity for TRAIL-R1 (DR4) and is thus a preferred mutation site. Preferred mutations are any which disrupt or ameliorate hydrogen bonding such as small, polar amino acids. Preferred is N199L or N199V, preferably N199V.

By way of explanation, hydrophobic: N199A/V/L, preferably N199V, is predicted to impact on protein-protein interactions, more so in TRAIL-R2 than TRAIL-R1 and is therby expected to confer specificity for TRAIL-R1. This mutation may also be used in combination with other mutations as described herein. Specificity may be greater for Leu and/or Val, which could form hydrophobic interactions with TRAIL to compensate for loss of hydrogen bond to backbone carbonyl of Cys [both -R1 and -R2] and sidechain of Arg [-R2]).

D267 and G160

From our model, we identify two further amino acids in TRAIL that may be critical for the interaction with TRAIL-R1, namely D267 and G160. Systematic substitution of these amino acid positions is therefore part of the present invention.

Considering D267, a larger negative residue such as D267E may enhance interaction with TRAIL-R1 (via K67), and is therefore preferred.

Gly160Leu may enhance TRAIL R1 interactions and is thus preferred. The reason is that of hydrophobic bulk: G160L may destabilise the interface between TRAIL-R2 and TRAIL by sterically preventing a salt bridge (R62 of TRAIL-R2 to E155 of TRAIL); additionally, this may further enhance interactions with TRAIL-R1 by forming additional hydrophobic interactions.

Further TRAIL Mutations

Supplementary mutations include targeting of interaction with lysine 67 in TRAIL R1. Without wishing to be bound by theory, it is thought that this residue may interact with Asp 267 in TRAIL. Thus, Asp 267 in TRAIL is a target site for further substitution according to the present invention as noted above.

Residues Asp203, Asp218 or Asp269 may contribute to TRAIL/TRAIL-R1 interaction. Preferably one or more of these residues is mutated to Ala, or preferably to an amino acid other than Ala. Preferably Asp203Ala, Asp218Ala and Asp269Ala mutations are disclaimed from the present invention.

Asp203 is preferably left as Asp (wild-type). TRAIL-R1 and -R2 do not show differences at this region. Asp203Ala is preferably disclaimed from the invention.

Asp218 may form a salt bridge with H53 in TRAIL-R2 (equivalent -R1 residue is A53). Hydrophobic substitutions are thus preferred: D218A may weaken TRAIL/TRAIL-R2 interaction, and D218M may both weaken the TRAIL/TRAIL-R2 interaction and strengthen the TRAIL/TRAIL-R1 interaction. Thus, D218M is preferred.

Asp269 is preferably mutated to a larger negative residue: D269E may enhance TRAIL-R1 binding and weaken TRAIL-R2 binding. Thus D269E is preferred.

Mutation Combinations

Each of the mutations has been separately described above for ease of understanding. It must be noted that the present invention relates to the mutations individually, but also relates to the mutations in combination with one another in single TRAIL ligands. Each combination is embraced unless otherwise stated. Preferably all TRAIL ligands of the present invention comprise Tyr at position 189. Thus, combinations described or discussed herein preferably additionally comprise 189Y.

Particularly preferred is an Asn199Val mutation in combination with R191L.

Preferably positions 213 and 215 remain wild-type.

Preferably positions 191 and 201 are both mutated relative to wild type.

Preferably positions 199 and 201 are both mutated relative to wild type.

At position 201, preferably mutation is to an amino acid other than Lys (wild-type), preferably to an amino acid other than Arg or Lys (wild-type).

Preferably TRAIL mutants according to the present invention comprise a 199 single mutation or a 191 single mutation, preferably a 191 and 199 double mutation, preferably a 199 and 201 double mutation, preferably a 199 and 201 together with one of 213 or 215 triple mutation. Preferably TRAIL mutants according to the present invention comprise a 199 and 201 and 213 and 215 quadruple mutation. As stated above, preferably each of these additionally comprises 189Y.

When a 199/201 double mutant is used, preferably the amino acids are Val/Arg.

Further Features of TRAIL Mutants

The TRAIL mutants of the invention may be tagged such as FLAG-tagged or His-tagged, preferably His tagged, preferably with a 6His tag, for purification and for ease of preparation. Preferably the TRAIL mutants are used in untagged form.

Preferably TRAIL mutants are trimeric. His tagging does not adversely affect trimerisation.

Signalling Assays

When employing test systems in accordance with the present invention, it is important to assess signalling via TRAIL R1 or TRAIL R2 in terms of the biological signal. Different outcomes or effects such as triggering of apoptosis are not related to the level of TRAIL R1 or TRAIL R2 expression, but to the signal. Thus, it is important to employ a functional assay in order to determine if the signal is relevant. Naturally, if no DR4 is present on a particular cell or cell type then clearly there will be no DR4 signal. However, a high level of TRAIL R1 expression does not necessarily indicate a high level of TRAIL R1 signal. For example, K562 cells express high levels of TRAIL R1 but are still not sensitive to a TRAIL-R1 specific antibody.

DR4 Selectivity/DR4 Signalling

DR4 selectivity means a preference of the ligand for DR4 receptor. Preferably this is a specificity for the DR4 receptor ie. preferably a DR4 selective ligand will complex only with DR4 (and preferably not with DR5).

One test for DR4 selectivity is binding in vivo in the cellular context. Preferably this is tested via death inducing signalling complex (DISC) data. In this regard, reference is made to the examples section, in particular to FIG. 2A (Ramos cells/TRAIL R1) and FIG. 2B (Jurkat cells/TRAIL R2). Preferably a DR4 selective ligand binds only DR4.

Another test can be conducted with R1/R2 specific blocking antibodies. These antibodies are applied in order to determine whether they neutralise the effect of a particular ligand. If an R1 (DR4) blocking antibody nleutralises the effect of a particular ligand but a R2 (DR5) blocking antibody does not neutralise the effect, then that ligand is DR4 selective.

Another test involves the assay of the effect in R1- (DR4-) only cells. This is the preferred test for DR4 selectivity as discussed herein. Preferably the ligand is applied to an R1-only signalling cell. Preferably the cell is a Ramos cell. Preferably the read-out is functional activity, preferably death-inducing activity (apoptosis). This is crucial to the test—mere binding is not as robust an indicator as functional induction of death. Thus, preferably a DR4 selective ligand according to the present invention induces death in a DR4 only signalling cell, such as a Ramos cell. More preferably said ligand also does not induce death in a DR5 only signalling cell, such as a Jurkat cell.

It is to be noted that the invention preferably deals with a selectivity effect, rather than just a bias or a specificity effect. The criteria which are preferably used to ascertain selectivity are: the ability to selectively isolate an active TRAIL-R1 receptor complex (DISC) and the ability of specific TRAIL-R1 blocking antibodies to neutralise the activity of the mutated TRAIL-R1 specific ligands. The same tests may be carried out in connection with TRAIL-R2 for comparative purposes, substituting R2 reagents as appropriate.

Sequence characteristics for DR4 selectivity are discussed herein above.

Sensitisation/Combination Treatments

Preferably treatment with TRAIL is in combination with sensitising agent. In terms of administration, combination may mean simultaneous, sequential or together. Sequential may mean sensitiser followed by TRAIL, or may mean TRAIL followed by sensitiser. The temporal gap between administration (if any) may be any suitable gap determined for practical or therapeutic reasons.

HDAC Inhibitors

Preferably the sensitising agent is a HDACi. Preferably the HDACi is selected from the group consisting of depsipeptide, Trichostatin A (TSA) and valproate. Preferably the HDACi is valproate. Previously valproate has been known as an anti-convulsive and as a treatment for conditions such as epilepsy. It is surprisingly disclosed herein that valproate finds application in leukaemic disorders such as CLL.

In this way, a TRAIL mutant may be used which is inactive or active at advantageously low levels in non-target tissue. However, by co-treating the target tissue with sensitising agent such as HDACi, TRAIL activity in that tissue is advantageously enhanced thereby producing therapeutically useful levels of cell death. Preferably at least one of the TRAIL and sensitising treatments are directed to the target tissue, preferably both are directed to the target tissue. Targeting may mean retention/binding/activation/cleavage into active form or other means of directing the agent to the target tissue following non-directed administration, or targeting may mean a local or restricted form of administration which physically directs the material to the target tissue.

Preferably TRAIL treatments are preceded by HDACi treatment. This advantageously kills cells which may not be killed by prior art techniques. For example, following HDACi-pre-treatment, the TRAIL-R1 specific mutant can kill cells not killed by the TRAIL-R1 Ab (HGS-ETR1). This study has been carried out in a cell line. This indicates that the range of activity of the ligand(s) may be broader than that of the Abs. Without wishing to be bound by theory, this effect may be due to somewhat different mechanisms by which the ligand and Ab induce apoptosis.

A HDACi is a preferred sensitising agent in connection with tumour producing cancers. A HDACi is a particularly preferred sensitising agent in connection with CLL.

Within the class of HDAC inhibitors, agents that preferentially target Class I HDAC's are preferred for sensitisation.

Agents that only specifically inhibit Class II HDAC's do not sensitize to TRAIL-induced apoptosis to a high enough level and are therefore preferably excluded from the present invention. Thus, Class I HDAC inhibitors are preferred.

Within Class I HDACi's, inhibitors of HDAC 1 and/or HDAC 2 are the most preferred.

Other Sensitising Agents

Proteasome inhibitors may be used as sensitising agents eg. PS-341 (bortezomib), MG132 or others.

Existing therapeutic agents may be used as sensitising agents eg. Doxorubicin, 5FU, Cisplatin, Flavopiridol, CD20 (Rituximab), Resveratrol or others.

Sensitisation of CLL cells (and indeed other primary tumors that signal via TRAIL-R1) may be achieved by upregulating TRAIL-R1 receptor levels. This may be carried out using chemotherapeutic agents that target AP1 or following HBV infection (eg. as described in Guan et al, Oncogene. 2002, 21:3121-9; Guan et al, J Cell Physiol. 2001,188:98-105; Janssen et al, J Hepatol. 2003, 39:414-20).

Preferably AP1 transcription is upregulated to sensitise cells.

Other means of sensitisation include modulation of p53.

Other means of sensitisation include radiotherapy. Preferably treatment of the invention is combined with radiotherapy to enhance the sensitivity to TRAIL.

Other sensitising agents include HepV virus, PKC inhibitors (PKCi's), and proteasome inhibitors, paying appropriate attention to the cellular context in each case.

Diseases

We have demonstrated that primary cells from both CLL and MCL patients signal primarily through TRAIL-R1 and thus the invention finds particular application in these disorders. The invention further finds application in other primary tumors of haematological lineage including Non-Hodgkin's Lymphoma (NHL), particularly B-cell NHL, and in other disorders whose cells signal primarily through TRAIL-R1.

The relative involvement of TRAIL-R1/R2 may thus be dependent on the cell lineage, or alternatively differences may exist between primary cells and immortalised cell lines. In this respect, most of the data generated supporting a primary role for TRAIL-R2 signalling has been in the context of immortalised cell lines rather than primary cells. However, there are some reports using cell lines that also support a potentially key role for TRAIL-R1 in certain non-haematological malignancies. These include prostate, NSCLC, colon and renal tumors as well as transformed human keratinocytes. Thus, the invention also finds application in treatment of these disorders.

Preferably the invention is applied to cells of the haematopoietic lineage such as non-Hodgkin's lymphoma, multiple myeloma, acute myeloid leukaemia or other cells such as the Jurkat cell line, Ramos cell line, T-cells or B-cells or their derivates. Preferably the invention is applied to cells of the B-cell lineage.

Verification should preferably be carried out in primary tumour cells since many cultured cell lines may preferentially signal through DR5.

In a broad aspect the invention relates to the use of a DR4 selective TRAIL in the treatment of a disorder whose cells signal primarily through TRAIL-R1.

Pharmaceutical Compositions

The present invention also provides a pharmaceutical composition comprising a therapeutically effective amount of the TRAIL and/or sensitising agent(s) of the present invention and a pharmaceutically acceptable carrier, diluent or excipient (including combinations thereof).

The pharmaceutical compositions may be for human or animal usage in human and veterinary medicine and will typically comprise any one or more of a pharmaceutically acceptable diluent, carrier, or excipient. Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), solubilising agent(s).

Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used.

There may be different composition/formulation requirements dependent on the different delivery systems. By way of example, the pharmaceutical composition of the present invention may be formulated to be administered using a mini-pump or by a mucosal route, for example, as a nasal spray or aerosol for inhalation or ingestable solution, or parenterally in which the composition is formulated by an injectable form, for delivery, by, for example, an intravenous, intramuscular or subcutaneous route. Alternatively, the formulation may be designed to be administered by a number of routes.

Where the agent is to be administered mucosally through the gastrointestinal mucosa, it should be able to remain stable during transit though the gastrointestinal tract; for example, it should be resistant to proteolytic degradation, stable at acid pH and resistant to the detergent effects of bile.

Where appropriate, the pharmaceutical compositions can be administered by inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or they can be injected parenterally, for example intravenously, intramuscularly or subcutaneously. For parenteral administration, the compositions may be best used in the form of a sterile aqueous solution which may contain other substances, for example enough salts or monosaccharides to make the solution isotonic with blood. For buccal or sublingual administration the compositions may be administered in the form of tablets or lozenges which can be formulated in a conventional manner.

For some embodiments, the TRAIL/sensitising agents of the present invention may also be used in combination with a cyclodextrin. Cyclodextrins are known to form inclusion and non-inclusion complexes with drug molecules. Formation of a drug-cyclodextrin complex may modify the solubility, dissolution rate, bioavailability and/or stability property of a drug molecule. Drug-cyclodextrin complexes are generally useful for most dosage forms and administration routes. As an alternative to direct complexation with the drug the cyclodextrin may be used as an auxiliary additive, e.g. as a carrier, diluent or solubiliser. Alpha-, beta- and gamma-cyclodextrins are most commonly used and suitable examples are described in WO-A-91/11172, WO-A-94/02518 and WO-A-98/55148.

The TRAIL may be prepared in situ in the subject being treated. In this respect, nucleotide sequences encoding said TRAIL may be delivered by use of non-viral techniques (e.g. by use of liposomes) and/or viral techniques (e.g. by use of retroviral vectors) such that the said TRAIL protein is expressed from said nucleotide sequence.

In a preferred embodiment, the pharmaceutical of the present invention is administered topically. Preferably the pharmaceutical is in a form that is suitable for topical delivery.

Administration

The term “administered” includes delivery by viral or non-viral techniques. Viral delivery mechanisms include but are not limited to adenoviral vectors, adeno-associated viral (AAV) vectors, herpes viral vectors, retroviral vectors, lentiviral vectors, and baculoviral vectors. Non-viral delivery mechanisms include lipid mediated transfection, liposomes, immunoliposomes, lipofectin, cationic facial amphiphiles (CFAs) and combinations thereof.

The components of the present invention may be administered alone but will generally be administered as a pharmaceutical composition—e.g. when the components are is in admixture with a suitable pharmaceutical excipient, diluent or carrier selected with regard to the intended route of administration and standard pharmaceutical practice.

For example, the components can be administered (e.g. orally or topically) in the form of tablets, capsules, ovules, elixirs, solutions or suspensions, which may contain flavouring or colouring agents, for immediate-, delayed-, modified-, sustained-, pulsed- or controlled-release applications.

If the pharmaceutical is a tablet, then the tablet may contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the agent may be combined with various sweetening or flavouring agents, colouring matter or dyes, with emulsifying and/or suspending agents and with diluents such as water, ethanol, propylene glycol and glycerin, and combinations thereof.

The routes for administration (delivery) include, but are not limited to, one or more of: oral (e.g. as a tablet, capsule, or as an ingestable solution), topical, mucosal (e.g. as a nasal spray or aerosol for inhalation), nasal, parenteral (e.g. by an injectable form), gastrointestinal, intraspinal, intraperitoneal, intramuscular, intravenous, intrauterine, intraocular, intradermal, intracranial, intratracheal, intravaginal, intracerebroventricular, intracerebral, subcutaneous, ophthalmic (including intravitreal or intracameral), transdermal, rectal, buccal, vaginal, epidural, sublingual.

In a preferred aspect, the pharmaceutical composition is delivered topically.

It is to be understood that not all of the components of the pharmaceutical need be administered by the same route. Likewise, if the composition comprises more than one active component, then those components may be administered by different routes.

If a component of the present invention is administered parenterally, then examples of such administration include one or more of: intravenously, intra-arterially, intraperitoneally, intrathecally, intraventricularly, intraurethrally, intrasternally, intracranially, intramuscularly or subcutaneously administering the component; and/or by using infusion techniques.

For parenteral administration, the component is best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

As indicated, the component(s) of the present invention can be administered intranasally or by inhalation and is conveniently delivered in the form of a dry powder inhaler or an aerosol spray presentation from a pressurised container, pump, spray or nebuliser with the use of a suitable propellant, e.g. dichlorodifluoromethane, trichlorofluoromethane, dichlorotetrafluoroethane, a hydrofluoroalkane such as 1,1,1,2-tetrafluoroethane (HFA 134A™) or 1,1,1,2,3,3,3-heptafluoropropane (HFA 227EA™), carbon dioxide or other suitable gas. In the case of a pressurised aerosol, the dosage unit may be determined by providing a valve to deliver a metered amount. The pressurised container, pump, spray or nebuliser may contain a solution or suspension of the active compound, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of the agent and a suitable powder base such as lactose or starch.

Alternatively, the component(s) of the present invention can be administered in the form of a suppository or pessary, or it may be applied topically in the form of a gel, hydrogel, lotion, solution, cream, ointment or dusting powder. The component(s) of the present invention may also be dermally or transdermally administered, for example, by the use of a skin patch. They may also be administered by the pulmonary or rectal routes. They may also be administered by the ocular route. For ophthalmic use, the compounds can be formulated as micronised suspensions in isotonic, pH adjusted, sterile saline, or, preferably, as solutions in isotonic, pH adjusted, sterile saline, optionally in combination with a preservative such as a benzylalkonium chloride. Alternatively, they may be formulated in an ointment such as petrolatum.

For application topically to the skin, the component(s) of the present invention can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water. Alternatively, it can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

The TRAIL and/or sensitising agent(s) of the present invention may be administered with one or more other pharmaceutically active substances. By way of example, the present invention covers the simultaneous, or sequential treatments with TRAIL and/or sensitising agent(s) according to the present invention and one or more steroids, analgesics, antivirals or other pharmaceutically active substance(s).

It will be understood that these regimes include the administration of the substances sequentially, simultaneously or together.

Due to the risk of destruction of TRAIL in the gastrointestinal tract, preferably TRAIL is not administered into the gastrointestinal tract or into the buccal or rectal cavities. Preferably TRAIL is administered by injection. Preferably TRAIL is formulated for administration by injection.

Sensitising agent such as HDACi may be administered by way of the gastrointestinal tract. In a preferred embodiment, the HDACi is sodium valproate and is preferably formulated for oral administration and is preferably administered orally.

Dose Levels

Typically, a physician will determine the actual dosage which will be most suitable for an individual subject. The specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy.

Depending upon the need, the agent may be administered at a dose of from 0.01 to 30 mg/kg body weight, such as from 0.1 to 10 mg/kg, more preferably from 0.1 to 1 mg/kg body weight.

Formulation

The component(s) of the present invention may be formulated into a pharmaceutical composition, such as by mixing with one or more of a suitable carrier, diluent or excipient, by using techniques that are known in the art.

Pharmaceutically Active Salt

The TRAIL and/or sensitising agent(s) of the present invention may be administered as a pharmaceutically acceptable salt. Typically, a pharmaceutically acceptable salt may be readily prepared by using a desired acid or base, as appropriate. The salt may precipitate from solution and be collected by filtration or may be recovered by evaporation of the solvent.

Further Advantages And Applications

Prior art ‘DR4’ TRAIL mutants have been shown to be inactive in DR4 cell lines. The present inventors have produced new mutants selective for the DR4 receptor. These mutant ligands find application in therapeutic and experimental systems, in particular in therapy of cancers such as chronic lymphocytic leukaemia (CLL), mantle cell lymphoma (MCL), and others which signal predominantly via DR4 (TRAIL R1).

Normal hepatocytes signal via TRAIL R2 (DR5) and so DR4 selective TRAIL ligands offer advantageously reduced liver toxicity.

The ligands of the invention thus have greater specificity and lower liver toxicity than prior art TRAIL mutants.

Preferably TRAIL mutants of the invention comprise amino acids 95-281 of TRAIL. Particularly preferred are TRAIL mutants TRAIL.R1-4 and TRAIL.R1-5 as shown in table 1. More preferred are these TRAIL mutants without the His tags.

TRAIL.R1-4 comprises 6His TRAIL 95-281 with 199Val, 201Arg, 213Trp and 215Asp.

TRAIL.R1-5 comprises 6His TRAIL 95-281 with 193Ser, 199Val, 201Arg, 213Trp and 215Asp.

The invention also relates to treatment of non-haematological cancers (e.g. non-B-cell derived cancers) such as breast cancer.

The invention also relates to TRAILs as described above for use as medicaments; use of TRAILs as described above for the manufacture of medicaments for cancer; and TRAILs as described above for use in the treatment of cancer.

The invention is now described by way of examples which are not intended to be limiting in nature but are provided to illustrate and help understand the invention.

### BRIEF DESCRIPTION OF THE FIGURES

FIG. 1 shows graphs and bar charts and photographs of visualised proteins.

FIG. 2 shows photographs of visualised proteins and a plot.

FIG. 3 shows annotated sequence comparisons and visual models of TRAIL structures.

FIG. 4 shows graphs and bar charts.

FIG. 5 shows photographs of blots.

FIG. 6 shows photographs of blots, photomicrographs of cells and a bar chart.

In slightly more detail:

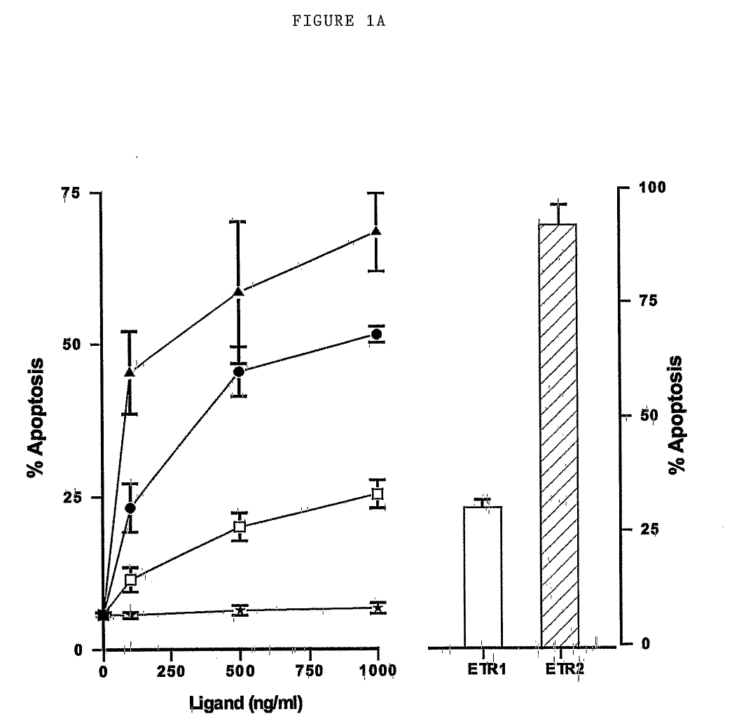

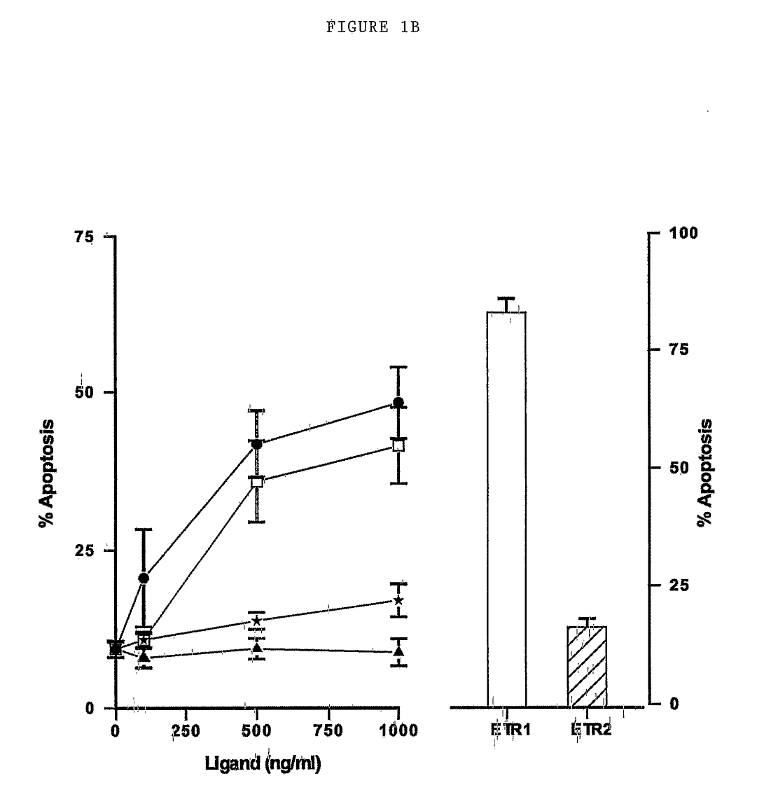

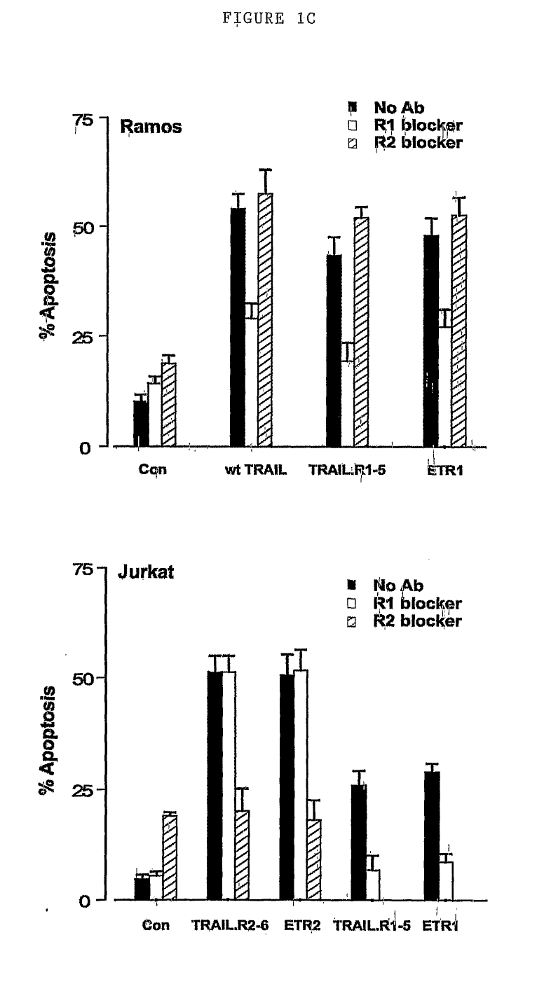

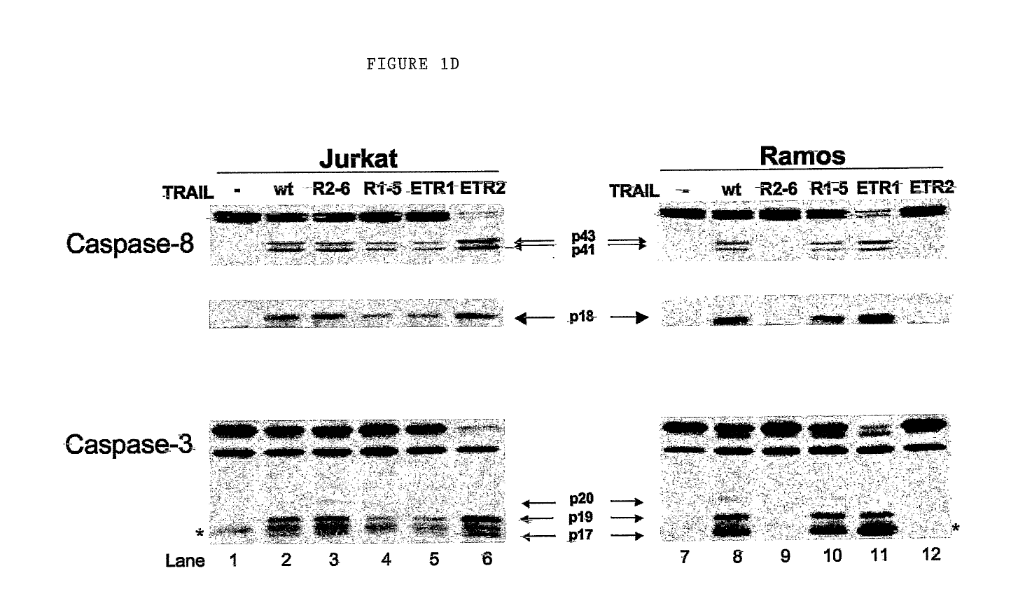

FIG. 1 shows cell type specific induction of apoptosis by TRAIL receptor selective mutants. A: Jurkat and B: Ramos cells were cultured for 4 h with the concentrations of wild type TRAIL (-), TRAIL.R1-5 (□-□), TRAIL.R2-6 (♦-♦) and TRAIL.R1-6 (*-*) and apoptosis measured. Cells were also exposed to HGS-ETR1 or HGSETR2 (1 μg ml⁻¹). C: Ramos or Jurkat cells were pre-incubated for 30 min either alone or with TRAIL-R1 blocking Ab or TRAIL-R2 neutralizing Ab (5 μg ml⁻¹). Ramos cells were then exposed for 4 h to either wt TRAIL, TRAIL.R1-5 (500 ng ml⁻¹), or HGS-ETR1 (1 pg ml⁻¹) and Jurkat cells were exposed to TRAIL.R2-6 (250 ng ml⁻¹), TRAIL.R1-5 (1 μg ml⁻¹) or HGS-ETR1 and ETR2 (1 μg ml⁻¹) and apoptosis assessed. D: Ramos and Jurkat cells were exposed to wt TRAIL or TRAIL mutants as described above and processing of caspase-3 and -8 determined by Western blot analysis. The asterisk indicates a non-specific band. The lower panel of the blot for detection of the caspase-8 p18 subunit was exposed for twice as long as the upper panel. Apoptosis was assessed by phosphatidylserine externalization and results expressed as the Means±SEM of at least 3 separate determinations.

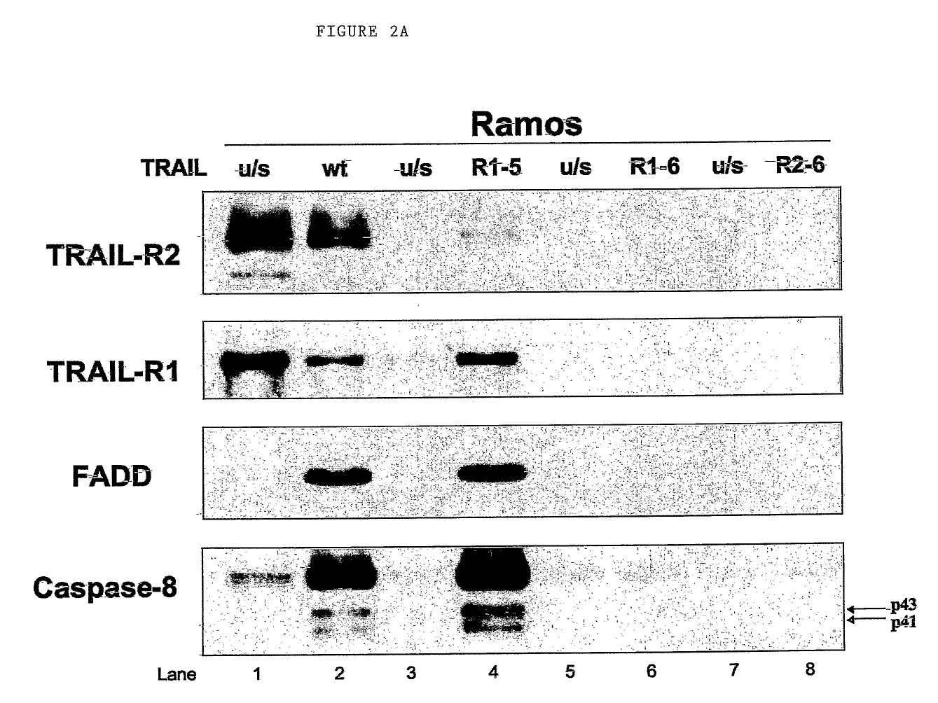

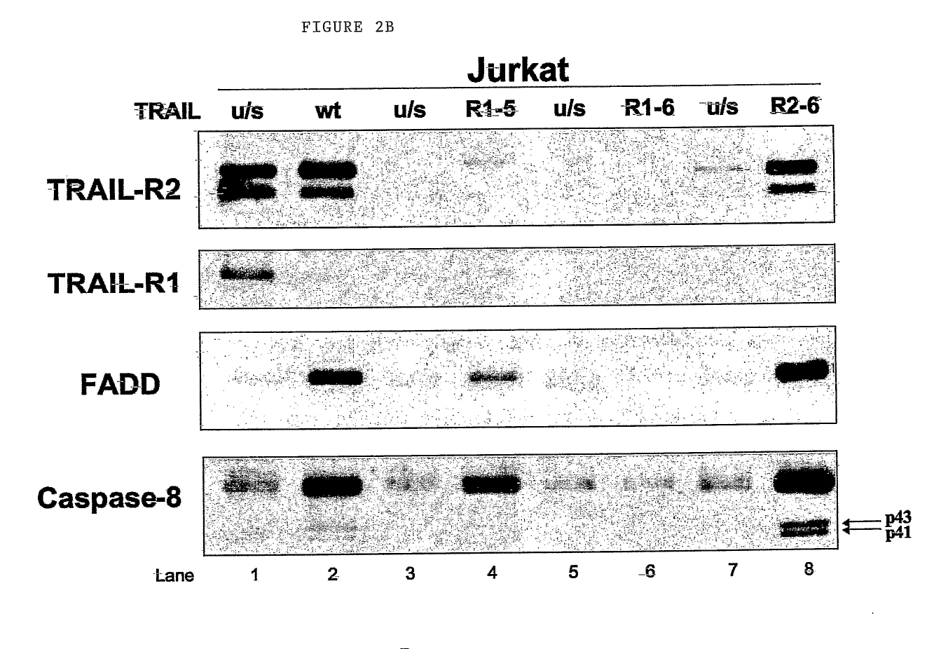

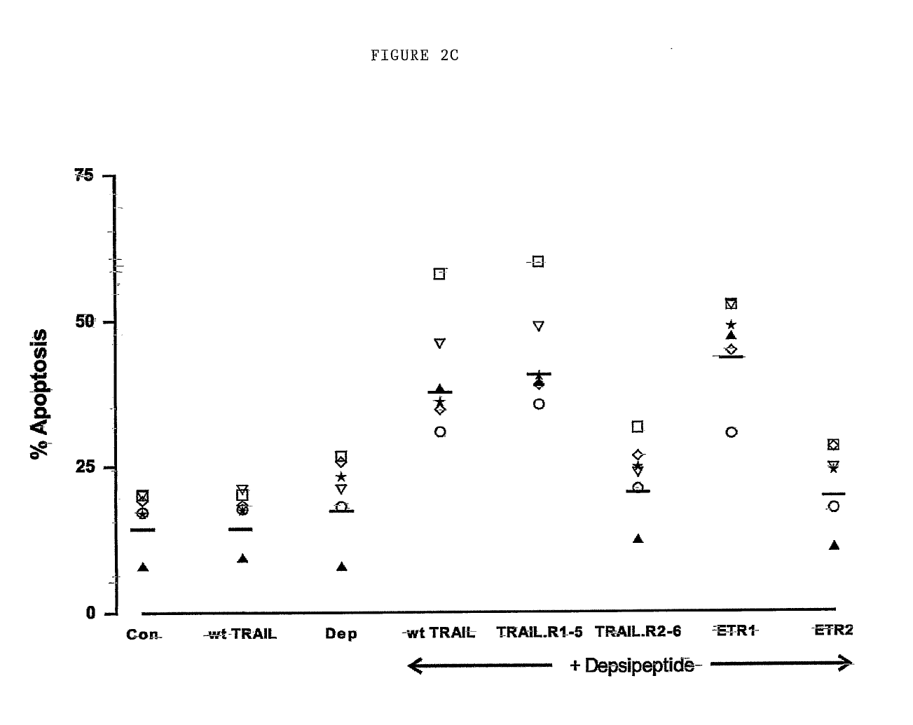

FIG. 2 shows that TRAIL mutants selectively induce DISC formation in different cell types. A: Ramos and B: Jurkat cells were exposed to 500 ng ml⁻¹ of the biotinylated forms of wt TRAIL (His-T), TRAIL.R1-5, the TRAIL-R1 selective mutant, TRAIL.R2-6, the TRAIL- R2 selective mutant, and TRAIL.R1-6, a related but inactive mutant. After exposure of the cells for 30 min, DISC complexes were isolated and analyzed for the indicated proteins by Western blotting. To provide an unstimulated receptor control (u/s), biotinylated wt or mutant TRAIL was added to lysate from untreated cells. C: Freshly isolated cells from patients with CLL were incubated for 16 h either alone or in the presence of depsipeptide (10 nM). Cells were then exposed for a further 6 h to wt TRAIL (100 ng ml⁻¹) or TRAIL.R1-5, TRAIL.R2-6, HGS-ETR1 (ETR1) or HGS-ETR2 (ETR2) at 1 μg ml⁻¹ and apoptosis assessed. Virtually identical results were obtained with TRAIL.R1-5 or TRAIL.R2-6 (100 ng ml⁻¹) but the higher concentration is shown to demonstrate the lack of activity of TRAIL.R2-6. The different symbols used indicate data from six individual patients and the solid line indicates the Mean. For clarity data from another patient with a high level of spontaneous apoptosis is omitted but showed the same trend.

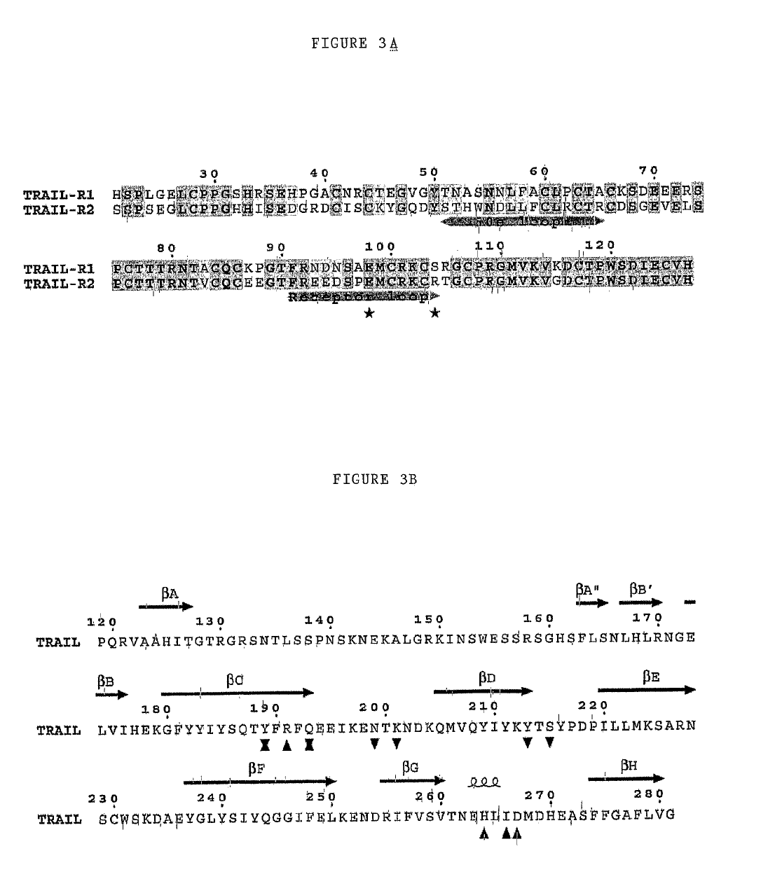

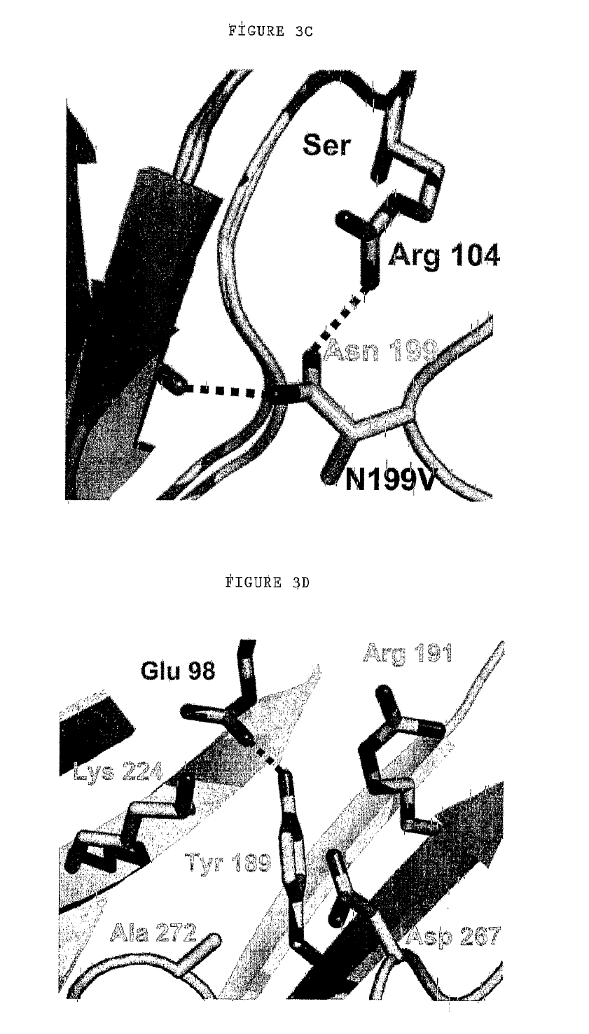

FIG. 3 shows a model of TRAIL/TRAIL-R1 complex and crystal structure of TRAIL/TRAIL-R2 complex. A: Sequence alignment of TRAIL-R1 and TRAIL-R2. Residue numbering shown is that for TRAIL-R2 (without its N-terminal signal peptide); residues on a cyan background are identical in the two structures and those on a yellow background are conserved; the role in TRAIL/TRAIL-R1/R2 interactions of residues Glu98 (denoted by blue star) and Arg(Ser)104 (red star) are discussed in the text. B: Sequence and crystallographic secondary structure (blue) of TRAIL, indicating the position of TRAIL.R1 (red down arrow) and TRAIL.R2 (blue up arrow) substitutions. C: Role of TRAIL Asn199 in TRAIL (yellow)/TRAIL-R1(cyan)/R2(green) interactions. The hydrogen bond present with both TRAIL-R1 and -R2 is shown as a black dashed line, and that present with only TRAIL-R2 as a red dashed line. The loss of these hydrogen bonds with the TRAIL substitution N199V is also illustrated. D: Role of TRAIL Tyr189 in TRAIL(yellow)/TRAIL-R1/R2(green) interactions. The hydrogen bond from this tyrosine to the conserved glutamate in TRAIL-R1/R2 is shown as a dashed line. Residues in TRAIL involved in hydrophobic interactions with Tyr189—interactions lost in Y189A substituted TRAIL—are also shown. Panels A and B were generated using ESPript (Gouet et al., 1999 Bioinformatics vol. 15, pp 305-308) and panels C and D using PyMol (DeLano, 2002: http://www.pymol.org).

### Examples

The Examples Make use of the Following Core Methods:

Lymphocyte Purification, Cell Lines and Culture

Ramos, an Epstein-Barr virus-negative Burkitt's lymphoma cell line, and Jurkat T cells (clone E6-1) were cultured as described (MacFarlane et al (2005 Cell Death and Differentiation vol 12 pages 773-782)). Blood samples were obtained from CLL patients, with patient consent and local ethical committee approval and CLL cells purified as described (MacFarlane et al (2002 Oncogene 21, 6809-6818). Cells were incubated for 16 h with depsipeptide (10 nM), provided by Dr. E. Sausville (NCI, USA). Cells were then cultured for a further 6 h with either HGSETR1 or HGS-ETR2, agonistic mAbs to TRAIL-R1 or -R2, respectively (Human Genome Sciences, Rockville, Md.), His-TRAIL-LE (a recombinant TRAIL with low endotoxin—Alexis Corporation, Nottingham, UK), His-TRAIL, a similar form of human recombinant TRAIL (MacFarlane et al (1997 J Biol Chem 272, 25417-25420)) but not further purified to reduce endotoxin levels or a mutant form of His-TRAIL as described (MacFarlane et al., 2005 ibid.). Cell lines were also pre-incubated for 30 min with a TRAIL-R1 blocking Ab and a TRAIL-R2 neutralizing Ab from Alexis and R&D systems, respectively, prior to exposure to TRAIL. Freshly isolated cells from patients with MCL were isolated, purified and incubated with different forms of TRAIL as described above for CLL cells. Samples were either analyzed immediately for apoptosis or stored at −80° C. for subsequent Western blotting. Other methods and reagents were as described (Inoue (2004 Cell Death Differ 11 Suppl 2, S193-206); MacFarlane et al., 2005 ibid).

Quantification of Apoptosis and Western Blot Analysis

Apoptosis was quantified by phosphatidylserine (PS) externalization in the presence of propidium iodide as described (MacFarlane et al., 2002 ibid.). Samples for Western blot analysis were prepared and caspase processing detected as described (Inoue et al., 2004 ibid.; MacFarlane et al., 2005 ibid.).

Synthesis of TRAIL Mutants

Mutants of His-TRAIL (residues 95-281) were generated using the Quik-Change site-directed mutagenesis kit (Stratagene, Calif., USA), confirmed by DNA sequencing, and then expressed in E. coli and purified as described previously (MacFarlane et al., 1997 ibid.).

DISC Analysis

Ramos or Jurkat cells (5×10⁷ cells per treatment) were exposed to biotin-tagged recombinant His-TRAIL (biotinylated TRAIL) or one of its mutant forms (500 ng ml⁻¹) for 30 min and then DISC formation assessed as described previously (MacFarlane et al., 2002 ibid.).

Modelling the TRAIL/TRAIL-R1 Complex

We modeled the TRAIL/TRAIL-R1 complex based on the crystal structure of the TRAIL/TRAIL-R2 complex. First, homology modeling (‘Modeller’) was used to produce a structural model of TRAIL-R1 based on the structure of TRAIL-R2. The TRAIL/TRAIL-R1 complex was then modeled by superposing the backbone atoms of the model of TRAIL-R1 onto the corresponding atoms of TRAIL-R2 in complex with TRAIL.

### Example 1

### Exemplary and Comparitive TRAIL Mutant Sequences

The following table shows wild-type TRAIL sequence at selected positions compared to prior art (‘Genentech’) and TRAIL mutants of the invention. Particularly preferred TRAIL mutants of the invention are TRAIL.R1-4 and TRAIL.R1-5.

| | 189 | 191 | 193 | 199 | 201 | 209 | 213 | 215 |
|---|---|---|---|---|---|---|---|---|
| Wt | Y | R | Q | N | K | Y | Y | S |
| Genentech | A | R | S | V | R | Y | W | D |
| (= FLAG- | | | | | | | | |
| Apo2L.DR4- | | | | | | | | |
| 8) | | | | | | | | |
| TRAIL.R1-5 | Y | R | S | V | R | Y | W | D |
| TRAIL.R1-4 | Y | R | Q | V | R | Y | W | D |

Furthermore, the TRAIL mutants of this example are longer than the prior art mutants since they contain residues 95-281 of TRAIL, and the Genentech version comprises only residues 114-281.

The TRAIL.R1-5 mutant only differs from the Genentech mutant at one amino acid (=residue 189). The mutant of the invention retains the wild type tyrosine whilst the Genentech mutant has an alanine substitution. The Genentech mutant shows selective binding for TRAIL R1 (DR4) by in vitro binding assays, but has no signalling activity in cells that signal via R1.

We show that although the Genentech mutant may show selective binding to R1, it does not have any R1 specific activity i.e. it does not induce apoptosis in cells that signal via R1. Furthermore, we have shown that mutant TRAIL.R1-5 does indeed induce R1 selective apoptotic activity. This is an unexpected effect of the ligands of the present invention, since the prior art teaches that substitution of Tyr-189 with Ala is important for DR4 selectivity.

TRAIL.R1-4 also shows good R1 selective activity, reinforcing the finding that a tyrosine at position 189 is important for R1 selective apoptotic activity.

### Example 2

### TRAIL Receptor Selective Mutants Signal to Apoptosis via TRAIL-R1 in Primary Lymphoid Malignancies

Overview

Based on studies using agonistic monoclonal antibodies (Mabs), we demonstrate that primary chronic lymphocytic leukemia (CLL) cells appear to signal primarily through TRAIL-R1 despite expressing TRAIL-R2 on the cell surface. We have synthesized mutant forms of TRAIL specific for TRAIL-R1 or TRAIL-R2. The selectivity of these mutants to induce apoptosis in cell lines is due to their selective binding to their cognate receptors resulting in apoptosis via formation of a death inducing signaling complex (DISC). Using these mutants we conclusively demonstrate that CLL and mantle cell lymphoma cells signal apoptosis almost exclusively through TRAIL-R1. These data confirm that the two TRAIL death receptors can signal independently and show that DR4 receptor-specific mutant forms of TRAIL have therapeutic applications.

Apoptotic Activity of TRAIL Receptor Selective Mutants

Recently, a novel phage display approach was utilized to select TRAIL mutants selective for binding to TRAIL-R1 or -R2, based on their affinities for the appropriate receptor-Fc. Only mutants selective for binding to TRAIL-R2 induced apoptosis in several cell lines leading the authors to conclude that signaling via TRAIL-R2 rather than TRAIL-R1 was more important for the induction of apoptosis (Kelley et al (2005 J Biol Chem Volume 280, pages 2205-2212)). However, we show that CLL cells signal almost entirely through TRAIL-R1. To resolve this discrepancy, we synthesized the two mutants, TRAIL.R1-6 and TRAIL.R2-6 (Table 1), reported to be selective for TRAIL-R1 or -R2, respectively and assessed their ability to induce apoptosis in Ramos and Jurkat cells, which signal predominantly through TRAIL-R1 and TRAIL-R2, respectively (MacFarlane et al (2005 Cell Death and Differentiation volume 12 pages 773-782)). Both HGS-ETR2 and TRAIL.R2-6, the TRAIL-R2 selective mutant, potently induced apoptosis in Jurkat but not in Ramos cells, as assessed by phosphatidylserine (PS) externalization (FIGS. 1A and B). These data support the conclusion that TRAIL.R2-6 (Table 1) is a selective TRAIL-R2 mutant in agreement with previous work. However TRAIL.R1-6, the proposed TRAIL-R1 selective mutant (Kelley et al., 2005), was inactive in Jurkat cells as expected but also was largely inactive in Ramos cells (FIGS. 1A & B and Table 1). We had predicted that this mutant would be active in Ramos cells, having reasoned that its observed inactivity (Kelley et al., 2005) was due to the use of cell lines that signaled primarily through TRAIL-R2 rather than TRAIL-R1. Clearly the loss of activity was due to the substitution of either some or all of the six target amino acid residues in TRAIL.R1-6. To test this, we synthesized TRAIL mutants with intermediate substitutions between the wild type (wt) protein and TRAIL.R1-6 (Table 1) and tested their activity in Ramos and Jurkat cells with the aim of obtaining mutants that would selectively induce apoptosis in Ramos cells. Initially we tested TRAIL.R1-2 as these two amino acid substitutions, Tyr213Tip and Ser215Asp, were included in all the TRAIL-R1 mutants in the phage display library because they had a similar affinity to TRAIL-R1-Fc but a 10-fold lower affinity for TRAIL-R2-Fc (Kelley et al., 2005). However, this mutant had a very similar activity to wt TRAIL in both Ramos and Jurkat cells (Table 1) suggesting that it was neither specific for TRAIL-R1 nor R2. Next we tested the TRAIL.R1-4 mutant, which induced similar levels of apoptosis to wt TRAIL in Ramos cells but exhibited a decreased activity in Jurkat cells (Table 1), suggesting a degree of selectivity for TRAIL-R1. An additional substitution at Tyr189Ala to yield TRAIL.R1-5a resulted in a mutant that lost all activity to TRAIL-R2 but also lost much of its activity to TRAIL-R1, as determined by its complete inactivity on Jurkat cells and markedly diminished activity on Ramos cells (Table 1). This led us to examine the effects of the single amino acid substitution Tyr189Ala alone. Compared to wt TRAIL, TRAIL.R1-1 exerted a decreased ability to induce apoptosis in both Ramos and Jurkat cells (Table 1). As the Tyr189Ala substitution in the TRAIL.R1-5a mutant resulted in such a loss of activity on Ramos cells, we chose to omit this substitution from the original TRAIL.R1-6. The resulting mutant, TRAIL.R1-5, retained most of the activity of wt TRAIL on Ramos cells but had lost much of its activity on Jurkat cells (Table 1). Further studies showed that this mutant demonstrated a very similar concentration-response to wt TRAIL in Ramos cells but was much less active than wt TRAIL in Jurkat cells (FIGS. 1A and B). Thus this mutant exhibited the requisite properties of inducing apoptosis primarily by signaling through TRAIL-R1. The specificity of the TRAIL.R1-5 mutant to signal through TRAIL-R1 in Ramos cells was confirmed by the ability of a blocking antibody specific to TRAIL-R1 but not to TRAIL-R2 to inhibit apoptosis (FIG. 1C). The specificity of the TRAIL-R2 neutralizing Ab was evident from its ability to inhibit both TRAIL.R2-6 and HGS-ETR2-induced apoptosis in Jurkat cells (FIG. 1C).

Caspase Activity

To confirm that the mutants were inducing apoptosis, we examined their effects on the processing of caspase-8, the apical caspase in death receptor-induced apoptosis and caspase-3, an effector caspase. In Ramos cells, wt TRAIL, TRAIL.R1-5 and HGS-ETR1, but not TRAIL.R2-6 or HGSETR2, induced the processing of caspase-8 to its p43/41 forms and its p18 catalytically active large subunit as well as the processing of caspase-3 to its active large subunit, p19/17 (FIG. 1D). In marked contrast in Jurkat cells, wt TRAIL, TRAIL.R2-6 and HGS-ETR2 were the most potent both at inducing apoptosis (Table 1) and in inducing the processing of caspases-8 and -3 (FIG. 1D lanes 2, 3 and 6). Both TRAIL.R1-5 and HGS-ETR1 induced a small amount of caspase processing (FIG. 1D lanes 4 and 5) commensurate with their ability to induce low levels of apoptosis in Jurkat cells (FIG. 1A) and compatible with there being a small amount of residual TRAIL-R1 signaling. Our finding that in Jurkat cells the TRAIL-R1 blocking Ab blocked both the TRAIL.R1-5 and HGS-ETR1 but not TRAIL.R2-6 or HGS-ETR2-induced apoptosis provided support for this notion (FIG. 1C). Taken together these data demonstrate that TRAIL.R1-5 and TRAIL.R2-6 are relatively specific for TRAIL-R1 and TRAIL-R2, respectively.

DISC Formation Induced by TRAIL Mutant Proteins

As formation of an active DISC is often rate limiting in many forms of death receptor-induced apoptosis, we examined the abilities of the mutant proteins to form a DISC in both Ramos and Jurkat cells. Biotinylated wt TRAIL bound both TRAIL-R1 and -R2, and recruited FADD and caspase-8 to the native DISC in Ramos cells and caspase-8 was processed to its p43 and p41 forms (FIG. 2A lane 2). Neither the proposed TRAIL-R1 selective mutant, TRAIL.R1-6, (Kelley et al., 2005), nor the TRAIL-R2 selective mutant, TRAIL.R2-6, bound detectable amounts of TRAIL-R1 or -R2 and consequently failed to recruit FADD or caspase-8 to the DISC (FIG. 2A lanes 6 & 8) so explaining their inability to induce apoptosis (FIG. 1 and Table 1). However TRAIL.R1-5, one of our selective TRAIL-R1 mutants (FIG. 1 and Table 1), bound predominantly TRAIL-R1 together with a small amount of TRAIL-R2, recruited FADD and caspase-8 and the latter was processed to its p43/41 forms (FIG. 2A lane 4). Thus in Ramos cells mutants such as TRAIL.R1-5 selectively bind to TRAIL-R1, recruit FADD and then recruit and activate caspase-8 within the DISC. In contrast in Jurkat cells, wt TRAIL and TRAIL.R2-6, the TRAIL-R2 selective mutant, bound TRAIL-R2 extensively in the DISC and recruited FADD and caspase-8, which was also processed to its p43/41 forms (FIG. 2B lanes 2 and 8). The inactive mutant TRAIL.R1-6 bound neither TRAIL-R1 nor -R2 and did not recruit FADD or caspase-8 to the DISC (FIG. 2B lane 6). TRAIL.R1-5 bound small amounts of both TRAIL-R1 and -R2 as well as some FADD within the DISC together with a small amount of caspase-8, which was also partly processed (FIG. 2B lane 4). Taken together these data demonstrate the critical importance of formation of a DISC containing primarily TRAIL-R1 or TRAIL-R2 in Ramos and Jurkat cells, respectively. Thus small structural changes in the ligand permit its preferential binding to TRAIL-R1 or -R2, thereby determining its ability to form a DISC and induce apoptosis in an appropriate target cell.

Impact of Substitutions on the Interaction of TRAIL with TRAIL-R1

To gain insight into the impact of TRAIL mutations on the binding of TRAIL to TRAIL-R1 (Table 1), we produced a structural model of the TRAIL/TRAIL-R1 complex and compared it with the crystal structure of the TRAIL/TRAIL-R2 complex. The comparison was facilitated by the level of sequence similarity between the two proteins (TRAIL-R1 and TRAIL-R2 share 64 % amino acid sequence identity in their extracellular domains) (FIG. 3A). As previously observed, the interaction of TRAIL with TRAIL-R2 occurs through two main interaction patches containing residues important for high-affinity binding. The first interaction patch is a hydrophobic area near the top of the TRAIL/TRAIL-R2 complex, referred to as the 50s loop, and is conserved in many TNF superfamily members. The second patch is an area, referred to as the receptor loop close to the bottom of the complex near the cell membrane, which contains features specific for each individual family member and controls receptor selectivity (FIG. 3A).

The receptor loop, containing residues 91-104, interacts with a cluster of residues around Gln205 in TRAIL near the bottom of the trimer complex (FIG. 3B). Compared with wt TRAIL, the initial substitutions, Tyr213Trp and Ser215Asp, in TRAIL.R1-2 did not show any differential effects on either Ramos or Jurkat cells (Table 1). However a further two substitutions, namely Asn199Val and Lys201Arg, resulted in the mutant TRAIL.R1-4, which retained the activity of wt TRAIL to Ramos cells but lost some activity on Jurkat cells (Table 1), thereby showing some selectivity for signaling through TRAIL-R1 compared with TRAIL-R2. Analysis of the TRAIL/TRAIL-R1 (our model) and the TRAIL/TRAIL-R2 interface suggests that this small increase in TRAIL-R1 selectivity may be due to the substitution Asn199Val (but not Lys201Arg). In TRAIL/TRAIL-R2 this substitution is predicted to cause the loss of two hydrogen bonds (to the side chain of Arg104 and to the main chain carbonyl of Cys125). In contrast the Asn199Val substitution would result in the loss of only one inter-protein hydrogen bond in TRAIL/TRAIL-R1 since in TRAIL-R1 the equivalent to Arg104 is a much shorter Ser residue which cannot hydrogen bond to Asn199 (FIGS. 3A & C). The Tyr189Ala substitution was present in two TRAIL mutants, TRAIL.R1-6 and TRAIL.R1-5a, which both exhibited a marked loss of activity (Table 1). The importance of Tyr189 in the binding of TRAIL-R1 and -R2 can be rationalized through both direct and indirect effects. Tyr189 forms a hydrogen bond to a conserved Glu in both TRAIL-R1 and -R2, corresponding to Glu98 in TRAIL-R2. The substitution Tyr189Ala removes this hydrogen bond in both the TRAIL/TRAIL-R1 and -R2 complexes (FIG. 3D). Substitution of Tyr189Ala will also result in the removal of hydrophobic interactions to Arg191, Asp267, Ala272 and Lys224 close to the surface of TRAIL (FIG. 3D). Thus this substitution may indirectly affect ligand binding by distorting the surface of TRAIL.

### Example 3

### TRAIL-R1 but not TRAIL-R2 Selective Mutants Induce Apoptosis in Cancer Cells

The cancer cells of this example are those of haematopoietic malignancies such as CLL cells and MCL cells. We disclose that TRAIL induces apoptosis in CLL cells by signaling through TRAIL-R1 but not TRAIL-R2. The synthesis of selective TRAIL mutants enables us to demonstrate this point unequivocally. Depsipeptide sensitized CLL cells to TRAIL-induced apoptosis (FIG. 2C). Most importantly depsipeptide also sensitized CLL cells to TRAIL.R1-5, the TRAIL-R1 selective mutant, but not to TRAIL.R2-6, the TRAIL-R2 selective mutant (FIG. 2C). Furthermore depsipeptide sensitized CLL cells to HGS-ETR1, the agonistic TRAIL-R1 Ab but not to HGS-ETR2, the TRAIL-R2 agonistic Ab (FIG. 2C).

We also obtained three samples from patients with mantle cell lymphoma (MCL), an incurable and aggressive disease that accounts for ˜6% of all non-Hodgkin's lymphoma. There are no standard treatments for MCL and the prognosis is very poor particularly for the blastoid variant. MCL cells were isolated and exposed to the different forms of TRAIL after pre-treatment for 16 h with depsipeptide (5 nM). Essentially similar results to CLL cells were obtained with primary MCL cells. Spontaneous apoptosis (23.4±2.9%) was not increased by wt TRAIL (25.5±3.8%) but was increased by depsipeptide (35.4±5%). Depsipeptide sensitized MCL cells to both HGS-ETR1 (68.2±6.1%) and TRAIL.R1-5 (54.3±9.1%) but not to HGS-ETR2 (38.3±4.3%) or TRAIL.R2-6 (35.5±3.1%). Taken together these data demonstrate unequivocally that TRAIL signals to apoptosis primarily by activating TRAIL-R1 but not TRAIL-R2 in haematological malignancies including both CLL and MCL cells.

By using TRAIL-R1 or TRAIL-R2 selective mutants, we have now unequivocally demonstrated that freshly isolated CLL and MCL cells, obtained directly from patients, signal to apoptosis almost exclusively via TRAIL-R1 and not TRAIL-R2. Whether other tumor subtypes' signalling of apoptosis preferentially through one or other TRAIL receptor can advantageously be determined in the same manner. These results clearly have significant implications for rational therapy with TRAIL or its agonistic Abs. If primary tumors, such as CLL or MCL, signal primarily through TRAIL-R1, then according to the present invention preparations of TRAIL that signal almost exclusively through TRAIL-R2, such as HGS-ETR2 (Human Genome Sciences) or Apo2L/TRAIL (Genentech) should be avoided and DR4 selective TRAIL according to the present invention should be used instead.

Our data highlight, that prior to initiating therapy, ie. in a diagnostic setting, it is critical to determine whether primary tumor cells signal via TRAIL-R1 or -R2. Furthermore our data demonstrate that small structural changes in TRAIL can allow preferential binding to either TRAIL-R1 or TRAIL-R2 with subsequent formation of a TRAIL-R1 or -R2 DISC followed by recruitment of FADD and caspase-8. Clearly such mutated ligands find application in therapy.

TABLE 1
Apoptosis inducing abilities of various TRAIL mutants

| | | % PS⁺cells (c) | |
|---|---|---|---|
| His-TRAIL (a) | Amino acid changes (b) | Ramos | Jurkat |

| | | | | | | | | | | | | |
|---|---|---|---|---|---|---|---|---|---|---|---|---|
| - (control) | | | | | | | | | | | 9.3 ± 1.3 | 5.7 ± 0.4 |
| wild type | 189 | 191 | 193 | 199 | 201 | 213 | 215 | 264 | 266 | 267 | 41.8 ± 5.2 | 45.5 ± 4.1 |
| | Tyr | Arg | Gln | Asn | Lys | Tyr | Ser | His | Ile | Asp | | |
| TRAIL.R1-6 | Ala | — | Ser | Val | Arg | Trp | Asp | — | — | — | 13.7 ± 1.3 | 6.4 ± 0.8 |
| TRAIL.R1-2 | — | — | — | — | — | Trp | Asp | — | — | — | 42.2 ± 3.1 | 46.6 ± 3.1 |
| TRAIL.R1-4 | — | — | — | Val | Arg | Trp | Asp | — | — | — | 43.5 ± 2.5 | 25.0 ± 2.6 |
| TRAIL.R1-5a | Ala | — | — | Val | Arg | Trp | Asp | — | — | — | 18.9 ± 0.7 | 6.6 ± 0.4 |
| TRAIL.R1-1 | Ala | — | — | — | — | — | — | — | — | — | 30.6 ± 2.9 | 24.9 ± 2.2 |
| TRAIL.R1-5 | — | — | Ser | Val | Arg | Trp | Asp | — | — | — | 35.9 ± 6.4 | 20.0 ± 2.3 |
| TRAIL.R2-6 | Gln | Lys | Arg | — | — | — | — | Arg | Leu | Gln | 9.4 ± 1.6 | 58.4 ± 11.6 |

a Mutants were produced from His-tagged TRAIL (95-281). Labelling of the mutant as R1 or R2 indicates it was synthesized with specificity for TRAIL-R1 or -R2 respectively. The last number of the mutant indicates the number of amino acid substitutions compared to wt TRAIL
^{b}Amino acid changes relative to the wild type TRAIL are shown in bold. The dash indicates no change in amino acid from wild type TRAIL.
c Apoptosis was measured in the indicated cell type by phosphatidylserine (PS) externalization after exposure for 4 h to TRAIL or its mutant (500 ng ml⁻). The results are expressed as Mean ± SEM of at least three separate detenninations.

### Example 4

### Receptor Selective TRAIL Mutants and Application to Diverse Cancers

In this example we present further preferred mutant TRAIL ligands. We further demonstrate the application of the invention to non-haematological cancers (e.g. non-B-cell derived cancers) such as breast cancer.

Materials and Methods

Ramos and Jurkat T cells (clone E6-1) were cultured as described (MacFarlane et al, 2005 ibid.). The breast tumour cell line, MCF-7 (clone F43) was cultured as described previously (MacFarlane et al., 2000 J. Cell Biol. 148, 1239-1254). Cells were cultured for either 4 h (Ramos and Jurkat) or 6 h (MCF-7) with His-TRAIL (MacFarlane et al, 1997 J Biol Chem 272, 25417-25420), HGS-ETR1 or HGS-ETR2 (Human Genome Sciences), or a mutant form of His-TRAIL (MacFarlane et al, 2005 ibid.). Samples were analyzed for apoptosis, Western blotting, or TRAIL DISC activation as described (MacFarlane et al, 2005 ibid.; MacFarlane et al. 2002 Oncogene 21, 6809-6818). Mutants of His-TRAIL (residues 95-281) were generated using the Quik-Change site-directed mutagenesis kit (Stratagene, Calif.), confirmed by DNA sequencing, expressed in E. coli and purified as described (MacFarlane et al, 1997 ibid.). For siRNA targeting of TRAIL-R1/-R2, MCF-7 cells were seeded into six-well plates immediately prior to effectene-mediated RNAi transfection with either a negative control siRNA (30 nM, Ambion, #4611), or preannealed siRNA oligonucleotides for TRAIL-R1 or TRAIL-R2 (30 nM, Ambion), with the sequence and validity described (Ren et al, 2004 Mol. Biol. Cell vol 15 pp 5064-5074). After 40 h of transfection the cells were collected and total TRAIL-R1 or TRAIL-R2 levels assessed. Where indicated, RNAi transfected MCF-7 cells were either left untreated or exposed to wt-TRAIL and apoptosis assessed by the extent of Bax activation, determined using the conformation specific Bax MAb, clone 3 (Dewson et al, 2003 Oncogene vol 22 pp 2643-54).

Further TRAIL-R1 Selective TRAIL Mutants

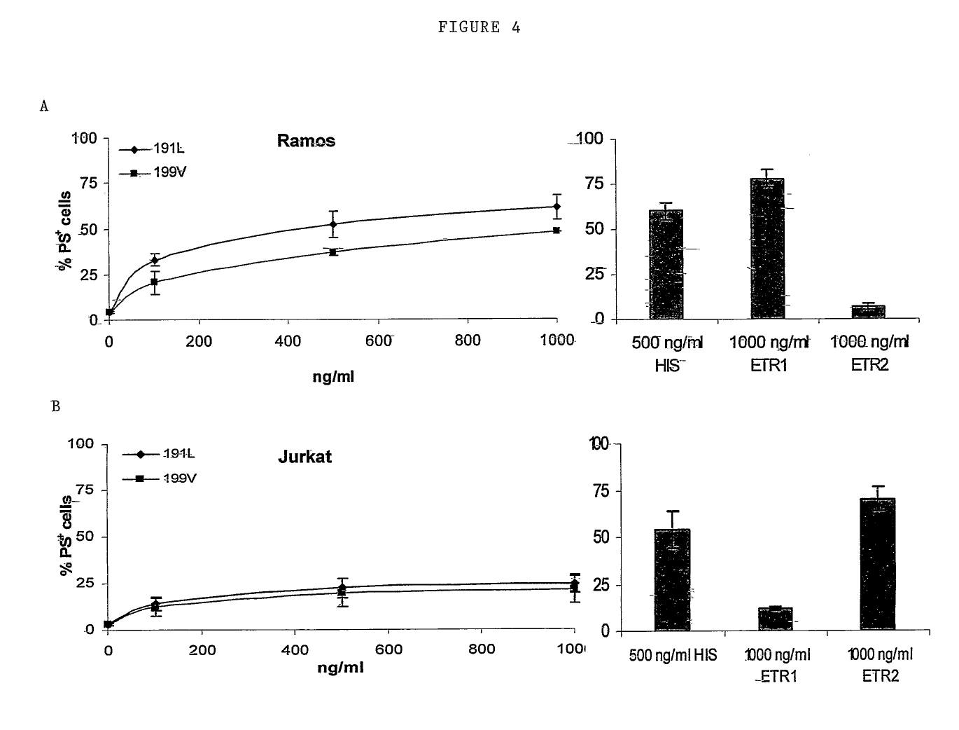

In this example we demonstrate that single amino acid substitutions in TRAIL can confer specificity for TRAIL -R1. Referring to FIG. 4: Ramos cells (A) and Jurkat cells (B) were cultured with increasing concentrations of Arg191Leu or Asn199Val single mutants of His-TRAIL (100-100 ng/ml) or His-TRAIL (500 ng/ml), ETR1 or ETR2 (1000 ng/ml) for 4 h. Apoptosis was quantified by measuring the % PS+ cells and results shown in FIG. 4 are the Mean±SEM of three independent experiments. Thus the biological importance TRAIL residues R191 and N199 in TRAIL-R1 selectivity is shown. Furthermore, it is shown that TRAIL R191L and TRAIL N199V are TRAIL-R1 selective TRAIL mutants.

Diverse Cancer Cells Respond to DR4-selective TRAILs

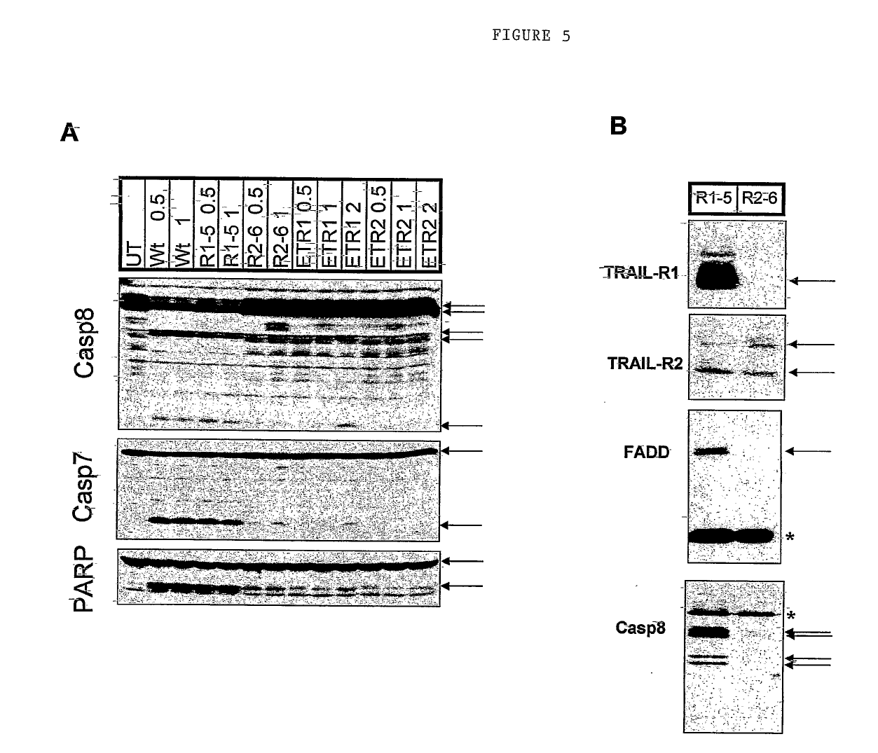

Use of receptor-selective sTRAIL mutants according to the present invention reveals that the breast tumour cell line MCF-7 signals to apoptosis via TRAIL-R1. Referring to FIG. 5: (A) MCF-7 cells were cultured with increasing concentrations of wt TRAIL, TRAIL.R1-5, TRAIL.R2-6 (500-1000 ng/ml), or ETR1, ETR2 (500-2000 ng/ml) for 6 h. Apoptosis was quantified by measuring the processing of Caspase-8, Caspase-7 and PARP by Western blot analysis. Caspase-8 was processed to its p43/p41 forms and to its p18 large subunit, caspase-7 to its p19 large subunit, and PARP to its p85 cleavage product. (B) The selectivity of TRAIL.R1-5 in MCF-7 cells was further confirmed by analysis of TRAIL DISC activation, which revealed that only TRAIL.R1-5, the TRAIL-R1-selective mutant, resulted in recruitment and processing Caspase-8 at the DISC. Results shown are from one experiment representative of three independent experiments.

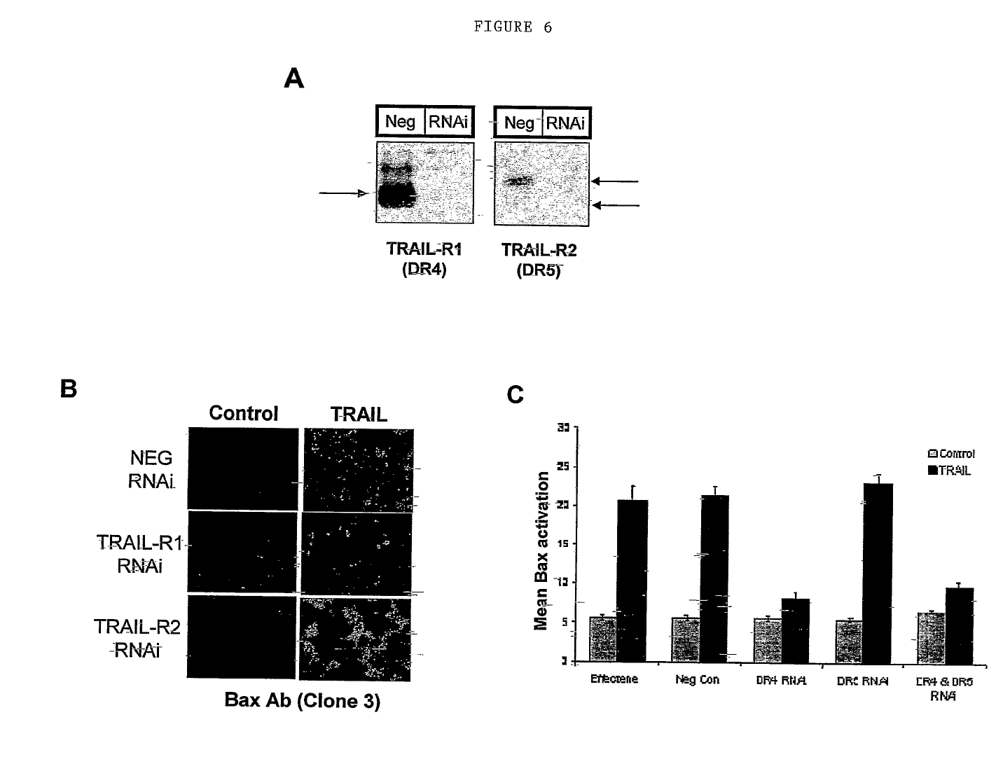

siRNA targeting of TRAIL-R1 and TRAIL-R2 also reveals that sTRAIL signals to apoptosis via TRAIL-R1 in the breast tumour cell line MCF-7. MCF-7 cells were transfected with either effectene alone, or effectene in the presence of a negative control siRNA (30 nM), or preannealed siRNA oligonucleotides for TRAIL-R1 or TRAIL-R2 (30 nM). Referring to FIG. 6: (A) After 40 h of transfection the cells were collected and total TRAIL-R1 or TRAIL-R2 levels assessed. (B) RNAi transfected MCF-7 cells were exposed to wt-TRAIL and apoptosis assessed by the extent of Bax activation, determined using the conformation specific Bax MAb, clone 3. Results shown are from one experiment representative of three independent experiments. (C) The extent of Bax activation in the presence and absence of the indicated siRNA oligonucleotides was assessed in three independent experiments. Results shown are the Mean±SEM.

These data show that breast cancer cells, such as the MCF7 cell line, signal through DR4 and thus positively demonstrate that DR4 signalling is important in diverse cancers as well as haematopoietic derived cancers. These findings therefore further illustrate industrial applications for the DR4 specific (DR4 selective) mutant TRAILs of the invention.

All publications mentioned in the above specification are herein incorporated by reference. Various modifications and variations of the described methods and system of the present invention will be apparent to those skilled in the art without departing from the scope of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

1. A TRAIL which selectively signals through DR4, comprising Y at position 189.

2. A TRAIL according to claim 1 further comprising
(i) 191L; or
(ii) 199V; or
(iii) 193S.

3. A TRAIL according to claim 1 further comprising 201R, 213W and 215D.

4. A TRAIL according to claim 1 which comprises sequence corresponding to at least amino acids 95-281 of TRAIL.

5. (canceled)

6. (canceled)

7. (canceled)

8. A method of treating a cancer in a subject by administration of TRAIL which is capable of selectively signalling through DR4, said method comprising determining if the target cells of said subject signal via DR4, wherein if said target cells do signal via DR4 then a TRAIL which of selectively signals through DR4 is administered.

9. The method according to claim 8, further comprising administering a sensitizing agent.

10. The method according to claim 9 wherein said sensitising agent is a HDAC inhibitor.

11. The method according to claim 10 wherein said HDAC inhibitor is valproate.

12. (canceled)

13. A method for treatment of a B-cell malignancy in a subject, the method comprising administering to said subject a TRAIL selectively signals through DR4.

44. The method of claim 13 wherein said B-cell malignancy is selected from the group consisting of B-cell non-Hodgkin's lymphoma, mantle cell lymphoma and chronic lymphocytic leukaemia.

15. The method according to claim 13 further comprising administering to said subject a histone deacetylase inhibitor (HDACi).

16. The method according to claim 15 wherein the TRAIL is administered after the HDACi is administered.

17. The method according to claim 16 wherein the TRAIL is administered at least 8 hours after the HDACi is administered.

18. The method according to claim 16 wherein the TRAIL is administered at least 16 hours after the HDACi is administered.

19. The method according to claim 15 wherein the HDACi is administered over a period of about 8-16 hours.

20. The method according to claim 13 wherein the TRAIL is administered over a period of about 4 hours.

21. A method of inducing formation of death inducing signalling complex (DISC) in a haematological malignant cell comprising contacting said cell with a histone deacetylase inhibitor (HDACi) and a TRAIL which selectively signals through DR4.

22. A method of inducing caspase activation in a haematological malignant cell comprising contacting said cell with a histone deacetylase inhibitor (HDACi) and a TRAIL which selectively signals through DR4.

23. The method according to claim 15 wherein the HDACi is selected from the group consisting of depsipeptide, Trichostatin A (TSA) and valproate.

24. The method according to claim 23 wherein the HDACi is valproate.

25. The method according to claim 24 wherein the valproate is sodium valproate.

26. The TRAIL according to claim 1 further comprising a non-peptide polymer.

27. The TRAIL according to claim 26 wherein said polymer is selected from the group consisting of polyethylene glycol, polypropylene glycol and polyoxyalkylene.

28. A kit comprising a TRAIL which selectively signals through DR4 and a sensitising agent.

29. (canceled)


SEQUENCE LISTING (9 sequences)

SEQ ID NO: 1 - protein, 281 aa, Homo sapiens [sequence omitted]

SEQ ID NO: 2 - protein, 187 aa, Artificial
  TRAIL mutant, His-TRAIL
TSEETISTVQEKQQNISPLVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 3 - protein, 187 aa, Artificial
  TRAIL mutant, TRAIL.R1-6
TSEETISTVQEKQQNISPLVRERGPQRVAAHITGTRGRSNTLSSPNSKNEKALGRKINSWESSRSGHSFLSNLHLRNGELVIHEKGFYYIYSQTAFRFSEEIKEVTRNDKQMVQYIYKWTDYPDPILLMKSARNSCWSKDAEYGLYSIYQGGIFELKENDRIFVSVTNEHLIDMDHEASFFGAFLVG

SEQ ID NO: 4 - protein, 187 aa, Artificial
  TRAIL mutant, TRAIL.R1-2
TSEETISTVQEKQQNISPLVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKWTDYPDPILLMKSARNSCWSKDAEYGLYSIYQGGIFELKENDRIFVSVTNEHLIDMDHEASFFGAFLVG

SEQ ID NO: 5 - protein, 187 aa, Artificial
  TRAIL mutant, TRAIL.R1-4
TSEETISTVQEKQQNISPLVRERGPQRVAAHITGTRGRSNTLSSPNSKNEKALGRKINSWESSRSGHSFLSNLHLRNGELVIHEKGFYYIYSQTYFRFQEEIKEVTRNDKQMVQYIYKWTDYPDPILLMKSARNSCWSKDAEYGLYSIYQGGIFELKENDRIFVSVTNEHLIDMDHEASFFGAFLVG

SEQ ID NO: 6 - protein, 187 aa, Artificial
  TRAIL mutant, TRAIL.R1-5a
TSEETISTVQEKQQNISPLVRERGPQRVAAHITGTRGRSNTLSSPNSKNEKALGRKINSWESSRSGHSFLSNLHLRNGELVIHEKGFYYIYSQTAFRFQEEIKEVTRNDKQMVQYIYKWTDYPDPILLMKSARNSCWSKDAEYGLYSIYQGGIFELKENDRIFVSVTNEHLIDMDHEASFFGAFLVG

SEQ ID NO: 7 - protein, 187 aa, Artificial
  TRAIL mutant, TRAIL.R1-1
TSEETISTVQEKQQNISPLVRERGPQRVAAHITGTRGRSNTLSSPNSKNEKALGRKINSWESSRSGHSFLSNLHLRNGELVIHEKGFYYIYSQTAFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 8 - protein, 187 aa, Artificial
  TRAIL mutant, TRAIL.R1-5
TSEETISTVQEKQQNISPLVRERGPQRVAAHITGTRGRSNTLSSPNSKNEKALGRKINSWESSRSGHSFLSNLHLRNGELVIHEKGFYYIYSQTYFRFSEEIKEVTRNDKQMVQYIYKWTDYPDPILLMKSARNSCWSKDAEYGLYSIYQGGIFELKENDRIFVSVTNEHLIDMDHEASFFGAFLVG

SEQ ID NO: 9 - protein, 187 aa, Artificial
  TRAIL mutant, TRAIL.R2-6
TSEETISTVQEKQQNISPLVRERGPQRVAAHITGTRGRSNTLSSPNSKNEKALGRKINSWESSRSGHSFLSNLHLRNGELVIHEKGFYYIYSQTQFKFREEIKENTKNDKQMVQYIYKYTSYPDPILLMKSARNSCWSKDAEYGLYSIYQGGIFELKENDRIFVSVTNERLLQMDHEASFFGAFLVG